SEQUENCE LISTING (189 sequences)

SEQ ID NO: 1 - protein, 330 aa, Artificial Sequence
  Trastuzumab WT IgE_VH_CH1_CH2
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 2 - protein, 108 aa, Artificial Sequence
  Trastuzumab WT IgE_CH3
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 3 - protein, 110 aa, Artificial Sequence
  Trastuzumab WT IgE_CH4
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 4 - protein, 8 aa, Artificial Sequence
  Trastuzumab IgE Loop 1
FDLFIRKS

SEQ ID NO: 5 - protein, 5 aa, Artificial Sequence
  Trastuzumab IgE Loop 2
PVGTR

SEQ ID NO: 6 - protein, 7 aa, Artificial Sequence
  Trastuzumab IgE Loop 3a
ASPSQTV

SEQ ID NO: 7 - protein, 13 aa, Artificial Sequence
  Trastuzumab IgE Loop 3b
RAVHEAASPSQTV

SEQ ID NO: 8 - protein, 15 aa, Artificial Sequence
  WT IgG_Hinge
EPKSCDKTHTCPPCP

SEQ ID NO: 9 - protein, 110 aa, Artificial Sequence
  WT IgG_CH2
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 10 - protein, 107 aa, Artificial Sequence
  WT IgG_CH3
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 9 aa, Artificial Sequence
  IgG FcRn-binding Loop 1
KDTLMISRT

SEQ ID NO: 12 - protein, 5 aa, Artificial Sequence
  IgG FcRn-binding Loop 2
TVLHQ

SEQ ID NO: 13 - protein, 6 aa, Artificial Sequence
  IgG FcRn-binding Loop 3A
LHNHYT

SEQ ID NO: 14 - protein, 12 aa, Artificial Sequence
  IgG FcRn-binding Loop 3
SVMHEALHNHYT

SEQ ID NO: 15 - protein, 219 aa, Artificial Sequence
  IgE_CH3_CH4 containing IgG FcRn-binding Loop 1
DSNPRGVSAYLSRPSPKDTLMISRTPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 16 - protein, 218 aa, Artificial Sequence
  IgE_CH3_CH4 containing IgG FcRn-binding Loop 2
DSNPRGVSAYLSRPSPFDLFIRKSPTITCLVVDLAPSKGTVNLTWSRASGKPVNHSTRKEEKQRNGTLTVTSTLTVLHQDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 17 - protein, 217 aa, Artificial Sequence
  IgE_CH3_CH4 containing IgG FcRn-binding Loop 3a
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEALHNHYTQRAVSVNPGK

SEQ ID NO: 18 - protein, 217 aa, Artificial Sequence
  IgE_CH3_CH4 containing IgG FcRn-binding Loop 3b
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICSVMHEALHNHYTQRAVSVNPGK

SEQ ID NO: 19 - protein, 219 aa, Artificial Sequence
  IgE_CH3_CH4 containing IgG FcRn-binding Loop 1 + Loop 2
DSNPRGVSAYLSRPSPKDTLMISRTPTITCLVVDLAPSKGTVNLTWSRASGKPVNHSTRKEEKQRNGTLTVTSTLTVLHQDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 20 - protein, 218 aa, Artificial Sequence
  IgE_CH3_CH4 containing IgG FcRn-binding Loop 1 + Loop 3a
DSNPRGVSAYLSRPSPKDTLMISRTPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEALHNHYTQRAVSVNPGK

SEQ ID NO: 21 - protein, 218 aa, Artificial Sequence
  IgE_CH3_CH4 containing IgG FcRn-binding Loop 1 + Loop 3b
DSNPRGVSAYLSRPSPKDTLMISRTPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICSVMHEALHNHYTQRAVSVNPGK

SEQ ID NO: 22 - protein, 217 aa, Artificial Sequence
  IgE_CH3_CH4 containing IgG FcRn-binding Loop 2 + Loop 3a
DSNPRGVSAYLSRPSPFDLFIRKSPTITCLVVDLAPSKGTVNLTWSRASGKPVNHSTRKEEKQRNGTLTVTSTLTVLHQDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEALHNHYTQRAVSVNPGK

SEQ ID NO: 23 - protein, 217 aa, Artificial Sequence
  IgE_CH3_CH4 containing IgG FcRn-binding Loop 2 + Loop 3b
DSNPRGVSAYLSRPSPFDLFIRKSPTITCLVVDLAPSKGTVNLTWSRASGKPVNHSTRKEEKQRNGTLTVTSTLTVLHQDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICSVMHEALHNHYTQRAVSVNPGK

SEQ ID NO: 24 - protein, 218 aa, Artificial Sequence
  IgE_CH3_CH4 containing IgG FcRn Loop 1 + Loop 2 + Loop 3a
DSNPRGVSAYLSRPSPKDTLMISRTPTITCLVVDLAPSKGTVNLTWSRASGKPVNHSTRKEEKQRNGTLTVTSTLTVLHQDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEALHNHYTQRAVSVNPGK

SEQ ID NO: 25 - protein, 218 aa, Artificial Sequence
  IgE_CH3_CH4 containing IgG FcRn Loop 1 + Loop 2 + Loop 3b
DSNPRGVSAYLSRPSPKDTLMISRTPTITCLVVDLAPSKGTVNLTWSRASGKPVNHSTRKEEKQRNGTLTVTSTLTVLHQDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICSVMHEALHNHYTQRAVSVNPGK

SEQ ID NO: 26 - protein, 218 aa, Artificial Sequence
  IgE_CH3_CH4 3His
DSNPRGVSAYLSRPSPFDLFIRKSPTITCLVVDLAPSKGTVNLTWSRASGKPVNHSTRKEEKQRNGTLTVTSTLPVGHRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAAHPSHTVQRAVSVNPGK

SEQ ID NO: 27 - protein, 237 aa, Artificial Sequence
  IgE_CH4 plus IgG1 Hinge_CH2 (containing RS linker)
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 28 - protein, 344 aa, Artificial Sequence
  IgE_CH4 plus IgG1 Hinge_CH2_CH3 (containing RS linker)
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 675 aa, Artificial Sequence
  heavy chain of the IgE plus IgG1 Hinge_CH2 construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 30 - protein, 782 aa, Artificial Sequence
  heavy chain of the IgE plus IgG1 Hinge_CH2_CH3 construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 5 aa, Artificial Sequence
  IgE Cepsilon3 loop
PVGHR

SEQ ID NO: 32 - protein, 7 aa, Artificial Sequence
  IgE Cepsilon4 loop sequence 1
AHPSHTV

SEQ ID NO: 33 - protein, 13 aa, Artificial Sequence
  IgE Cepsilon4 loop sequence 2
RAVHEAAHPSHTV

SEQ ID NO: 34 - protein, 548 aa, Artificial Sequence
  heavy chain of the IgE 3His construct (WT IgE_VH_CH1_CH2 plus IgE_CH3_CH4 3His)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGHRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAAHPSHTVQRAVSVNPGK

SEQ ID NO: 35 - protein, 214 aa, Artificial Sequence
  light chain of the IgE 3His construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 8 aa, Artificial Sequence
  Alemtuzumab CDR H1
GFTFTDFY

SEQ ID NO: 37 - protein, 10 aa, Artificial Sequence
  Alemtuzumab CDR H2
IRDKAKGYTT

SEQ ID NO: 38 - protein, 12 aa, Artificial Sequence
  Alemtuzumab CDR H3
AREGHTAAPFDY

SEQ ID NO: 39 - protein, 6 aa, Artificial Sequence
  Alemtuzumab CDR L1
QNIDKY

SEQ ID NO: 40 - protein, 3 aa, Artificial Sequence
  Alemtuzumab CDR L2
NTN

SEQ ID NO: 41 - protein, 9 aa, Artificial Sequence
  Alemtuzumab CDR L3
LQHISRPRT

SEQ ID NO: 42 - protein, 5 aa, Artificial Sequence
  Atezolizumab CDR H1
DSWIH

SEQ ID NO: 43 - protein, 10 aa, Artificial Sequence
  Atezolizumab CDR H2
WISPYGGSTY

SEQ ID NO: 44 - protein, 7 aa, Artificial Sequence
  Atezolizumab CDR H3
RHWPGGF

SEQ ID NO: 45 - protein, 7 aa, Artificial Sequence
  Atezolizumab CDR L1
DVSTAVA

SEQ ID NO: 46 - protein, 6 aa, Artificial Sequence
  Atezolizumab CDR L2
SASFLY

SEQ ID NO: 47 - protein, 9 aa, Artificial Sequence
  Atezolizumab CDR L3
QQYLYHPAT

SEQ ID NO: 48 - protein, 5 aa, Artificial Sequence
  Avelumab CDR H1
SYIMM

SEQ ID NO: 49 - protein, 10 aa, Artificial Sequence
  Avelumab CDR H2
SIYPSGGITF

SEQ ID NO: 50 - protein, 9 aa, Artificial Sequence
  Avelumab CDR H3
IKLFTVTTV

SEQ ID NO: 51 - protein, 8 aa, Artificial Sequence
  Avelumab CDR L1
VGGYNYVS

SEQ ID NO: 52 - protein, 6 aa, Artificial Sequence
  Avelumab CDR L2
DVSNRP

SEQ ID NO: 53 - protein, 10 aa, Artificial Sequence
  Avelumab CDR L3
SSYTSSSTRV

SEQ ID NO: 54 - protein, 8 aa, Artificial Sequence
  Bevacizumab CDR H1
GYTFTNYG

SEQ ID NO: 55 - protein, 8 aa, Artificial Sequence
  Bevacizumab CDR H2
INTYTGEP

SEQ ID NO: 56 - protein, 16 aa, Artificial Sequence
  Bevacizumab CDR H3
AKYPHYYGSSHWYFDV

SEQ ID NO: 57 - protein, 6 aa, Artificial Sequence
  Bevacizumab CDR L1
QDISNY

SEQ ID NO: 58 - protein, 3 aa, Artificial Sequence
  Bevacizumab CDR L2
FTS

SEQ ID NO: 59 - protein, 9 aa, Artificial Sequence
  Bevacizumab CDR L3
QQYSTVPWT

SEQ ID NO: 60 - protein, 18 aa, Artificial Sequence
  Certolizumab CDR H2
GWINTYIGEPIYADSVKG

SEQ ID NO: 61 - protein, 11 aa, Artificial Sequence
  Certolizumab CDR H3
ARGYRSYAMDY

SEQ ID NO: 62 - protein, 11 aa, Artificial Sequence
  Certolizumab CDR L1
KASQNVGTNVA

SEQ ID NO: 63 - protein, 6 aa, Artificial Sequence
  Certolizumab CDR L2
SASFLY

SEQ ID NO: 64 - protein, 8 aa, Artificial Sequence
  Certolizumab CDR L3
QQYNIYPL

SEQ ID NO: 65 - protein, 8 aa, Artificial Sequence
  Cetuximab CDR H1
GFSLTNYG

SEQ ID NO: 66 - protein, 7 aa, Artificial Sequence
  Cetuximab CDR H2
IWSGGNT

SEQ ID NO: 67 - protein, 13 aa, Artificial Sequence
  Cetuximab CDR H3
ARALTYYDYEFAY

SEQ ID NO: 68 - protein, 6 aa, Artificial Sequence
  Cetuximab CDR L1
QSIGTN

SEQ ID NO: 69 - protein, 3 aa, Artificial Sequence
  Cetuximab CDR L2
YAS

SEQ ID NO: 70 - protein, 9 aa, Artificial Sequence
  Cetuximab CDR L3
QQNNNWPTT

SEQ ID NO: 71 - protein, 5 aa, Artificial Sequence
  Durvalumab CDR H1
RYWMS

SEQ ID NO: 72 - protein, 10 aa, Artificial Sequence
  Durvalumab CDR H2
NIKQDGSEKY

SEQ ID NO: 73 - protein, 10 aa, Artificial Sequence
  Durvalumab CDR H3
EGGWFGELAF

SEQ ID NO: 74 - protein, 8 aa, Artificial Sequence
  Durvalumab CDR L1
RVSSSYLA

SEQ ID NO: 75 - protein, 6 aa, Artificial Sequence
  Durvalumab CDR L2
DASSRA

SEQ ID NO: 76 - protein, 9 aa, Artificial Sequence
  Durvalumab CDR L3
QQYGSLPWT

SEQ ID NO: 77 - protein, 10 aa, Artificial Sequence
  Efalizumab CDR H1
GYSFTGHWMN

SEQ ID NO: 78 - protein, 20 aa, Artificial Sequence
  Efalizumab CDR H2
GIMIHPSDSETRYNQKFKDI

SEQ ID NO: 79 - protein, 16 aa, Artificial Sequence
  Efalizumab CDR H3
ARIGIYFYGTTYFDYI

SEQ ID NO: 80 - protein, 11 aa, Artificial Sequence
  Efalizumab CDR L1
RASKTISKYLA

SEQ ID NO: 81 - protein, 6 aa, Artificial Sequence
  Efalizumab CDR L2
SGSTLQ

SEQ ID NO: 82 - protein, 8 aa, Artificial Sequence
  Efalizumab CDR L3
QQHNEYPL

SEQ ID NO: 83 - protein, 11 aa, Artificial Sequence
  Omalizumab CDR H1
GYSITSGYSWN

SEQ ID NO: 84 - protein, 17 aa, Artificial Sequence
  Omalizumab CDR H2
ASITYDGSTNYADSVKG

SEQ ID NO: 85 - protein, 14 aa, Artificial Sequence
  Omalizumab CDR H3
ARGSHYFGHWHFAV

SEQ ID NO: 86 - protein, 15 aa, Artificial Sequence
  Omalizumab CDR L1
RASQSVDYDGDSYMN

SEQ ID NO: 87 - protein, 6 aa, Artificial Sequence
  Omalizumab CDR L2
AASYLE

SEQ ID NO: 88 - protein, 8 aa, Artificial Sequence
  Omalizumab CDR L3
QQSHEDPY

SEQ ID NO: 89 - protein, 10 aa, Artificial Sequence
  Panitumumab CDR H1
GGSVSSGDYY

SEQ ID NO: 90 - protein, 7 aa, Artificial Sequence
  Panitumumab CDR H2
IYYSGNT

SEQ ID NO: 91 - protein, 11 aa, Artificial Sequence
  Panitumumab CDR H3
VRDRVTGAFDI

SEQ ID NO: 92 - protein, 6 aa, Artificial Sequence
  Panitumumab CDR L1
QDISNY

SEQ ID NO: 93 - protein, 3 aa, Artificial Sequence
  Panitumumab CDR L2
DAS

SEQ ID NO: 94 - protein, 9 aa, Artificial Sequence
  Panitumumab CDR L3
QHFDHLPLA

SEQ ID NO: 95 - protein, 8 aa, Artificial Sequence
  Pertuzumab CDR H1
GFTFTDYT

SEQ ID NO: 96 - protein, 8 aa, Artificial Sequence
  Pertuzumab CDR H2
VNPNSGGS

SEQ ID NO: 97 - protein, 12 aa, Artificial Sequence
  Pertuzumab CDR H3
ARNLGPSFYFDY

SEQ ID NO: 98 - protein, 6 aa, Artificial Sequence
  Pertuzumab CDR L1
QDVSIG

SEQ ID NO: 99 - protein, 3 aa, Artificial Sequence
  Pertuzumab CDR L2
SAS

SEQ ID NO: 100 - protein, 9 aa, Artificial Sequence
  Pertuzumab CDR L3
QQYYIYPYT

SEQ ID NO: 101 - protein, 8 aa, Artificial Sequence
  Rituximab CDR H1
GYTFTSYN

SEQ ID NO: 102 - protein, 10 aa, Artificial Sequence
  Certolizumab CDR H1
GYVFTDYGMN

SEQ ID NO: 103 - protein, 8 aa, Artificial Sequence
  Rituximab CDR H2
IYPGNGDT

SEQ ID NO: 104 - protein, 3 aa, Artificial Sequence
  Rituximab CDR L2
ATS

SEQ ID NO: 105 - protein, 9 aa, Artificial Sequence
  Rituximab CDR L3
QQWTSNPPT

SEQ ID NO: 106 - protein, 8 aa, Artificial Sequence
  Trastuzumab CDR H1
GFNIKDTY

SEQ ID NO: 107 - protein, 8 aa, Artificial Sequence
  Trastuzumab CDR H2
IYPTNGYT

SEQ ID NO: 108 - protein, 13 aa, Artificial Sequence
  Rituximab CDR H3
ARSTYYGGDWFNV

SEQ ID NO: 109 - protein, 5 aa, Artificial Sequence
  Rituximab CDR L1
SSVSY

SEQ ID NO: 110 - protein, 13 aa, Artificial Sequence
  Trastuzumab CDR H3
SRWGGDGFYAMDY

SEQ ID NO: 111 - protein, 6 aa, Artificial Sequence
  Trastuzumab CDR L1
QDVNTA

SEQ ID NO: 112 - protein, 3 aa, Artificial Sequence
  Trastuzumab CDR L2
SAS

SEQ ID NO: 113 - protein, 9 aa, Artificial Sequence
  Trastuzumab CDR L3
QQHYTTPPT

SEQ ID NO: 114 - protein, 5 aa, Artificial Sequence
  Adalimumab CDR H1
DYAMH

SEQ ID NO: 115 - protein, 17 aa, Artificial Sequence
  Adalimumab CDR H2
AITWNSGHIDYADSVEG

SEQ ID NO: 116 - protein, 12 aa, Artificial Sequence
  Adalimumab CDR H3
VSYLSTASSLDY

SEQ ID NO: 117 - protein, 11 aa, Artificial Sequence
  Adalimumab CDR L1
RASQGIRNYLA

SEQ ID NO: 118 - protein, 7 aa, Artificial Sequence
  Adalimumab CDR L2
AASTLQS

SEQ ID NO: 119 - protein, 9 aa, Artificial Sequence
  Adalimumab CDR L3
QRYNRAPYT

SEQ ID NO: 120 - protein, 10 aa, Artificial Sequence
  Basiliximab CDR H1
GYSFTRYWMH

SEQ ID NO: 121 - protein, 17 aa, Artificial Sequence
  Basiliximab CDR H2
AIYPGNSDTSYNQKFEG

SEQ ID NO: 122 - protein, 8 aa, Artificial Sequence
  Basiliximab CDR H3
DYGYYFDF

SEQ ID NO: 123 - protein, 10 aa, Artificial Sequence
  Basiliximab CDR L1
SASSSRSYMQ

SEQ ID NO: 124 - protein, 7 aa, Artificial Sequence
  Basiliximab CDR L2
DTSKLAS

SEQ ID NO: 125 - protein, 7 aa, Artificial Sequence
  Basiliximab CDR L3
HQRSSYT

SEQ ID NO: 126 - protein, 10 aa, Artificial Sequence
  Belimumab CDR H1
GGTFNNNAIN

SEQ ID NO: 127 - protein, 17 aa, Artificial Sequence
  Belimumab CDR H2
GIIPMFGTAKYSQNFQG

SEQ ID NO: 128 - protein, 14 aa, Artificial Sequence
  Belimumab CDR H3
SRDLLLFPHHALSP

SEQ ID NO: 129 - protein, 11 aa, Artificial Sequence
  Belimumab CDR L1
QGDSLRSYYAS

SEQ ID NO: 130 - protein, 7 aa, Artificial Sequence
  Belimumab CDR L2
GKNNRPS

SEQ ID NO: 131 - protein, 11 aa, Artificial Sequence
  Belimumab CDR L3
SSRDSSGNHWV

SEQ ID NO: 132 - protein, 10 aa, Artificial Sequence
  Daclizumab CDR H1
GYTFTSYRMH

SEQ ID NO: 133 - protein, 17 aa, Artificial Sequence
  Daclizumab CDR H2
YINPSTGYTEYNQKFKD

SEQ ID NO: 134 - protein, 7 aa, Artificial Sequence
  Daclizumab CDR H3
GGGVFDY

SEQ ID NO: 135 - protein, 10 aa, Artificial Sequence
  Daclizumab CDR L1
SASSSISYMH

SEQ ID NO: 136 - protein, 7 aa, Artificial Sequence
  Daclizumab CDR L2
TTSNLAS

SEQ ID NO: 137 - protein, 9 aa, Artificial Sequence
  Daclizumab CDR L3
HQRSTYPLT

SEQ ID NO: 138 - protein, 6 aa, Artificial Sequence
  Infliximab CDR H1
IFSNHW

SEQ ID NO: 139 - protein, 10 aa, Artificial Sequence
  Infliximab CDR H2
RSKSINSATH

SEQ ID NO: 140 - protein, 7 aa, Artificial Sequence
  Infliximab CDR H3
NYYGSTY

SEQ ID NO: 141 - protein, 7 aa, Artificial Sequence
  Infliximab CDR L1
FVGSSIH

SEQ ID NO: 142 - protein, 7 aa, Artificial Sequence
  Infliximab CDR L2
KYASESM

SEQ ID NO: 143 - protein, 5 aa, Artificial Sequence
  Infliximab CDR L3
QSHSW

SEQ ID NO: 144 - protein, 10 aa, Artificial Sequence
  Natalizumab CDR H1
GFNIKDTYIH

SEQ ID NO: 145 - protein, 17 aa, Artificial Sequence
  Natalizumab CDR H2
RIDPANGYTKYDPKFQG

SEQ ID NO: 146 - protein, 14 aa, Artificial Sequence
  Natalizumab CDR H3
EGYYGNYGVYAMDY

SEQ ID NO: 147 - protein, 11 aa, Artificial Sequence
  Natalizumab CDR L1
KTSQDINKYMA

SEQ ID NO: 148 - protein, 7 aa, Artificial Sequence
  Natalizumab CDR L2
YTSALQP

SEQ ID NO: 149 - protein, 8 aa, Artificial Sequence
  Natalizumab CDR L3
LQYDNLWT

SEQ ID NO: 150 - protein, 12 aa, Artificial Sequence
  Palivizumab CDR H1
GFSLSTSGMSVG

SEQ ID NO: 151 - protein, 16 aa, Artificial Sequence
  Palivizumab CDR H2
DIWWDDKKDYNPSLKS

SEQ ID NO: 152 - protein, 10 aa, Artificial Sequence
  Palivizumab CDR H3
SMITNWYFDV

SEQ ID NO: 153 - protein, 10 aa, Artificial Sequence
  Palivizumab CDR L1
KCQLSVGYMH

SEQ ID NO: 154 - protein, 7 aa, Artificial Sequence
  Palivizumab CDR L2
DTSKLAS

SEQ ID NO: 155 - protein, 9 aa, Artificial Sequence
  Palivizumab CDR L3
FQGSGYPFT

SEQ ID NO: 156 - protein, 10 aa, Artificial Sequence
  Ranibizumab CDR H1
GYDFTHYGMN

SEQ ID NO: 157 - protein, 17 aa, Artificial Sequence
  Ranibizumab CDR H2
WINTYTGEPTYAADFKR

SEQ ID NO: 158 - protein, 13 aa, Artificial Sequence
  Ranibizumab CDR H3
YPYYYGTSHWFDV

SEQ ID NO: 159 - protein, 11 aa, Artificial Sequence
  Ranibizumab CDR L1
SASQDISNYLN

SEQ ID NO: 160 - protein, 7 aa, Artificial Sequence
  Ranibizumab CDR L2
FTSSLHS

SEQ ID NO: 161 - protein, 9 aa, Artificial Sequence
  Ranibizumab CDR L3
QQYSTVPWT

SEQ ID NO: 162 - protein, 8 aa, Artificial Sequence
  HMW-MAA CDR H1
GFTFSNYW

SEQ ID NO: 163 - protein, 10 aa, Artificial Sequence
  HMW-MAA CDR H2
IRLKSNNFGR

SEQ ID NO: 164 - protein, 13 aa, Artificial Sequence
  HMW-MAA CDR H3
TSYGNYVGHYFDH

SEQ ID NO: 165 - protein, 6 aa, Artificial Sequence
  HMW-MAA CDR L1
QNVDTN

SEQ ID NO: 166 - protein, 3 aa, Artificial Sequence
  HMW-MAA CDR L2
SAS

SEQ ID NO: 167 - protein, 9 aa, Artificial Sequence
  HMW-MAA CDR L3
QQYNSYPLT

SEQ ID NO: 168 - protein, 123 aa, Artificial Sequence
  HMW-MAA Variable Domain (Heavy Chain)
EQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSS

SEQ ID NO: 169 - protein, 107 aa, Artificial Sequence
  HMW-MAA Variable Domain (Light Chain)
DIELTQSPKFMSTSVCDRVSVTCKASQNVDTNVAWYQQKPGQSPEPLLFSASYRYTGVPDRFTGSGSGTDFTLTISNVQSEDLAEYFCQQYNSYPLTFGGGTKLEIK

SEQ ID NO: 170 - protein, 123 aa, Artificial Sequence; identical to SEQ ID NO: 168
  HMW-MAA VH

SEQ ID NO: 171 - protein, 107 aa, Artificial Sequence; identical to SEQ ID NO: 169
  HMW-MAA VL

SEQ ID NO: 172 - protein, 548 aa, Artificial Sequence
  Trastuzumab IgE Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 173 - protein, 782 aa, Artificial Sequence; identical to SEQ ID NO: 30
  Trastuzumab IgE-IgG-Fc Heavy Chain

SEQ ID NO: 174 - protein, 782 aa, Artificial Sequence
  Trastuzumab IgE-IgG-Fc C220S Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 782 aa, Artificial Sequence
  Trastuzumab IgG-IgG-Fc dFcRn Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 176 - protein, 782 aa, Artificial Sequence
  Trastuzumab IgG-IgG-Fc dFcRn C220S Heavy Chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 177 - protein, 214 aa, Artificial Sequence
  Kappa Trastuzumab Light Chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 549 aa, Artificial Sequence
  HMW-MAA IgE Heavy Chain
EVQLVQSGGGLVQPGGSLKLSCAVSGFTFSNYWMNWVRQAPGKGLEWVGEIRLKSNNFRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 179 - protein, 784 aa, Artificial Sequence
  HMW-MAA IgE IgG-Fc Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 784 aa, Artificial Sequence
  HMW-MAA IgE-IgG-Fc C220S Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 784 aa, Artificial Sequence
  HMW-MAA IgG-IgG-Fc dFcRn Heavy Chain
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSCDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 182 - protein, 784 aa, Artificial Sequence
  HMW-MAA IgG-IgG-Fc dFcRn C220S Heavy ChaiN
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSEPKSSDKTHTCPPCPAPELLGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 183 - protein, 214 aa, Artificial Sequence
  HMW-MAA Kappa Light Chain
DIQLTQSPSFLSASVGDRVTITCKASQNVDTNVAWYQQKPGKAPKPLLFSASYRYTGVPSRFSGSGSGTDFTLTISSLQPEDFATYFCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 122 aa, Artificial Sequence
  HMW-MAA Alternative Variable Domain (Heavy Chain)
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSS

SEQ ID NO: 185 - protein, 107 aa, Artificial Sequence
  HMW-MAA Alternative Variable Domain (Light Chain)
DIQLTQSPSFLSASVGDRVTITCKASQNVDTNVAWYQQKPGKAPKPLLFSASYRYTGVPSRFSGSGSGTDFTLTISSLQPEDFATYFCQQYNSYPLTFGGGTKVEIK

SEQ ID NO: 186 - protein, 551 aa, Artificial Sequence
  HMW-MAA heavy chain with 3 His mutation
EQVKLQQSGGGLVQPGGSMKLSCVVSGFTFSNYWMNWVRQSPEKGLEWIAEIRLKSNNFGRYYAESVKGRFTISRDDSKSSAYLQMINLRAEDTGIYYCTSYGNYVGHYFDHWGQGTTVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGHRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAAHPSHTVQRAVSVNPGK

SEQ ID NO: 187 - protein, 214 aa, Artificial Sequence
  HMW-MAA light chain
DIELTQSPKFMSTSVCDRVSVTCKASQNVDTNVAWYQQKPGQSPEPLLFSASYRYTGVPDRFTGSGSGTDFTLTISNVQSEDLAEYFCQQYNSYPLTFGGGTKLEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 550 aa, Artificial Sequence
  Alternative HMW-MAA heavy chain with 3His mutation
EVQLVQSGGGLVQPGGSLKLSCAVSGFTFSNYWMNWVRQAPGKGLEWVGEIRLKSNNFGRYYAESVKGRFTISRDDSKNTAYLQMNSLKTEDTAVYYCTSYGNYVGHYFDHWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGHRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAAHPSHTVQRAVSVNPGK

SEQ ID NO: 189 - protein, 214 aa, Artificial Sequence
  Alternative HMW-MAA light chain
DIQLTQSPSFLSASVGDRVTITCKASQNVDTNVAWYQQKPGKAPKPLLFSASYRYTGVPSRFSGSGSGTDFTLTISSLQPEDFATYFCQQYNSYPLTFGGGTKVEIKGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0380482 A1
(43) Pub. Date: Dec. 1, 2022

(54) IgE Antibody with FcRn binding

(71) Applicant: Epsilogen Ltd, London (GB)

(72) Inventors: Tim Wilson, London (GB); Kevin Fitzgerald, London (GB)

(21) Appl. No.: 17/764,854

(22) PCT Filed: Oct. 1, 2020
(86) PCT No.: PCT/EP2020/077609

§ 371 Date: Mar. 29, 2022

(30) Foreign Application Priority Data
Oct. 1, 2019 (GB) 1914165.4
Nov. 22, 2019 (GB) 1917059.6
Jun. 2, 2020 (GB) 2008248.3

(51) Int. Cl. C07K 16/32; C07K 16/28; A61P 35/00

(52) U.S. Cl. CPC C07K 16/32; C07K 16/283; A61P 35/00; C07K 2317/92; C07K 2317/732; C07K 2317/24; C07K 2317/524; C07K 2317/526; C07K 2317/66

(57) ABSTRACT

Described herein are hybrid antibodies targeted for use in the treatment of cancer. The antibodies have binding capabilities for Fcε receptors and the neonatal Fc receptor, which may be achieved e.g. by replacing sequences or amino acids in IgE constant domain with corresponding sequences and amino acids derived from IgG.

### FIELD OF THE INVENTION

The present invention lies in the design of synthetic (non-naturally occurring) hybrid antibodies, in particular hybrid IgE antibodies, together with their therapeutic use.

### BACKGROUND TO THE INVENTION

Immunoglobulin E (IgE) is a class of antibody (or immunoglobulin (Ig) “isotype”) that has only been found in mammals. IgE is synthesised by plasma cells. As with all antibody classes, monomers of IgE consist of two larger, identical heavy chains (ε chain) and two identical light chains (which are common to all antibody classes), with the ε chain containing four Ig-like constant domains (Cε1-Cε4).

It is the nature of the heavy chains that differentiates the different antibody classes, with those of the IgE class being larger and more heavily glycosylated than the heavy chains of the more common IgG class. Each antibody chain is comprised of a series of tandemly arranged immunoglobulin domains. The N-terminal domains (one each on the light and heavy chains) contain regions of highly variable sequence that enable binding to a huge range of antigens (the variable domains). The remaining domains consist of highly conserved so-called constant (Fc) domains.

One function of IgE is immunity to parasites such as helminths. IgE also has an essential role in type I hypersensitivity, which manifests in various allergic diseases, such as allergic asthma, most types of sinusitis, allergic rhinitis, food allergies, and specific types of chronic urticaria and atopic dermatitis. IgE also plays a pivotal role in responses to allergens, such as: anaphylactic drugs, bee stings, and antigen preparations used in desensitization immunotherapy.

Although IgE is typically the least abundant isotype, IgE levels in a normal (“non-atopic”) individual are only 0.05% of the Ig concentration, compared to 75% for the IgGs at 10 mg/ml, which are the isotypes responsible for most of the classical adaptive immune response and are capable of triggering the most powerful inflammatory reactions.

IgG is the main type of antibody found in blood and extracellular fluid, allowing it to control infection of body tissues. By binding many kinds of pathogens such as viruses, bacteria, and fungi, IgG protects the body from infection. IgG antibodies are large molecules with a molecular weight of about 150 kDa made of four peptide chains. Each molecule contains two identical class γ heavy chains of about 50 kDa and two identical light chains of about 25 kDa, thus a tetrameric quaternary structure. The two heavy chains are linked to each other and to a light chain each by disulphide bonds. The resulting tetramer has two identical halves which, together, form the Y-like shape. Each end of the fork contains an identical antigen binding site.

The structural differences confer different biological activities among the classes of antibody due to the panoply of effector cells and factors that bind to the different constant domains of each antibody class. The gamma chain of IgG binds to a broad family of receptors that include classical membrane-bound surface receptors, as well as atypical intracellular receptors and cytoplasmic glycoproteins. The membrane-bound surface receptors include FcγRI (CD64), FcγRIIa, FcγRIIb, FcγRIIIa (CD16) and FcγRIIIb. Similarly, the epsilon chain of IgE binds to a high affinity receptor, FccRI and a lower affinity receptor FcεRII. The differential expression of these various receptors on differing immune effector cells determines the type of immune response that can be generated by IgG and IgE.

Among the atypical FcγRs, the neonatal Fc receptor (FcRn) has gained notoriety given its intimate influence on IgG biology and its ability also to bind to albumin. FcRn functions as a recycling or transcytosis receptor that is responsible for maintaining IgG and albumin in the circulation, and bidirectionally transporting these two ligands across polarised cellular barriers. It has also been appreciated that FcRn acts as an immune receptor by interacting with and facilitating antigen presentation of peptides derived from IgG immune complexes (IC).

The neonatal Fc receptor (FcRn) belongs to the extensive and functionally divergent family of MHC molecules. Contrary to classical MHC family members, FcRn possesses little diversity and is unable to present antigens. Instead, through its capacity to bind IgG and albumin with high affinity at low pH, it regulates the serum half-lives of both of these proteins. IgG enjoys a serum half-life that is substantially longer than similarly-sized globular proteins, including IgE which does not bind to FcRn (approximately 21 days for IgG and <2 days for IgE). In addition, FcRn plays important role in immunity at mucosal and systemic sites through both its ability to affect the lifespan of IgG as well as its participation in innate and adaptive immune responses. FcRn expression is now recognised to be widespread, occurring throughout life and is expressed by a wide variety of parenchymal cell types in many different species. These include vascular endothelium (including the central nervous system), most epithelial cell types such as placental (syncytiotrophoblasts), epidermal (keratinocytes), intestinal (enterocytes), renal glomerular (podocytes), bronchial, mammary gland (ductal and acinar), retinal pigment epithelial cells, renal proximal tubular cells (PTC), hepatocytes, melanocytes, as well as cells of the choroid, ciliary body and iris in the eye. FcRn is also widely expressed by hematopoietic cells including monocytes, macrophages, dendritic cells (DC), neutrophils and B cells where, in contrast to polarised epithelial cells, it is detected in significant quantities on the cell surface (Zhu X et al (2001) J. Immunol. 166(5):3266-76).

Of the four IgG subclasses in humans (IgG1, IgG2, IgG3 and IgG4), binding affinity to FcRn ranges from 20 nM (IgG1) to 80 nM (IgG4) (West A P Jr, Bjorkman P J (2000) Biochemistry 39(32):9698-708). Structural studies have shown that FcRn binds to IgG with 1:1 or 2:1 stoichiometry under non-equilibrium or equilibrium conditions, respectively (Popov S. et al (1996) Mol. Immunol. 33(6):521-30; Sánchez L. M. et al (1999) Biochemistry 38(29):9471-6). FcRn binds independently to both sites of the IgG homodimer with identical affinity (Haberger M. et al (2015) mAbs 7:331-43), but that the avidity effect resulting from the 2:1 complex formation in known to be important for half-life extension.

Biochemical and crystallographic data indicate that upon binding at pH 6.0, neither FcRn nor IgG undergo major conformational changes. The key residues in IgG4 that are thought to impact binding to FcRn are Ile253, Ser254, Lys288, Thr307, Gln311, Asn434, and His435. In IgG1 it is the protonation of histidine residues in the Cγ2-Cγ3 hinge region which enable binding (Martin W. L. et al (2001) Molecular Cell 7:867-877). Due to their pKa, the histidine residues become protonated at pH ˜6 which allows for interaction with the FcRn residues Glu115 and Asp130. As the pH increases above 6, histidine protonation is gradually lost which explains the pH dependence of the interaction (Oganesyan V. et al supra; Raghavan M. et al (1995) Biochemistry 34:14649-57; Kim J.K. et al (1999) Eur J. Immunol. 29:2819-2825). This allows for the formation of salt bridges at the FcRn-Fc interface, specifically the acidic residues on the C-terminal portion of the α2 domain in FcRn (West et al supra, Martin et al supra, Vaughn D E, Bjorkman P J. (1998) Structure 6:63-73). In addition to the heavy chain interactions, β₂m also forms contacts with IgG through the Ile1 residue (Shields R. L. et al (2001) J. Biol. Chem. 276:6591-604). The FcRn binding site on IgG is distinct and distant from the binding site for classical FcγR which requires the glycosylation at the Asn297 residue of the Fc region of IgG (Tao M. H., Morrison S. L. (1989) J. Immunol. 143:2595-601).

Given the expanding use of monoclonal antibodies (mAb) as treatment in a range of human ailments including chronic inflammation, infections, cancer, autoimmune diseases, cardiovascular diseases and transplantation medicine, FcRn has emerged as major modifier of mAb efficacy (Chan A. C., Carter P. J. (2010) Nat. Rev. Immunol. 10:301-16; Weiner L. M. et al (2010) Nat. Rev. Immunol. 10:317-27). This is directly related to the persistence of the therapeutic antibody in the bloodstream, which in turn can increase localisation to the target site. To ensure long circulatory half-life of IgG, pH dependent binding and FcRn dependent recycling are crucial. Importantly, limited binding at neutral pH is required for proper release of IgG from cells and increasing the mAb affinity to FcRn at acidic pH correlates with half-life extension. Thus, IgG Fc engineering to optimise pH dependent binding to FcRn has been explored to tailor pharmacokinetics and increase IgG mAb half-life (Dall'Acqua W. F. et al (2006) J. Biol. Chem. 281:23514-24; Yeung Y. A. et al (2009) J. Immunol. 182:7663-1; Zalevsky J. et al (2010) Nat. Biotechnol. 28:157-9).

IgE is mostly known for its detrimental role in allergy, but several studies have long pointed towards a natural tumour surveillance function of this antibody isotype (Jensen-Jarolim E. et al (2008) Allergy 63: 1255-1266; Jensen-Jarolim E., Pawelec G. (2012) Cancer Immunol. Immunother. 61: 1355-1357). Pioneer studies with IgG and IgE antibodies of the same epitope specificity tested head-to-head revealed a higher potential of the IgE in terms of cytotoxicity (Gould H. J. et al (1999) Eur. J Immunol. 29: 3527-3537).

IgE has evolved to kill tissue-dwelling multicellular parasites, endowing it with several key features that make it ideal for use in the treatment of solid tumours, which also mostly reside in tissue. The epsilon constant region of IgE has a uniquely high affinity for its cognate receptor (FccRI) on the surfaces of immune effector cells including macrophages, monocytes, basophils and eosinophils Ka˜ 10¹⁰/M for FcεRI and Ka˜ 10⁸-10⁹/M for the CD23 trimer complex; Gould H. J., Sutton B. J. (2008) Nat. Rev. Immunol. 8: 205-217). This interaction is up to 10,000-fold greater than the affinity that the gamma chain of IgG has for its cognate receptors and this results in the majority of IgE molecules being permanently attached to the surface of immune effector cells (Fridman W. H. (1991) FASEB J. 5: 2684-2690). Therefore, the latter are primed and ready to destroy cells expressing the antigen recognised by the IgE. As a result, IgE is able to permeate tissues more effectively than IgG and stimulate significantly greater levels of both antibody-dependent cell-mediated phagocytosis (ADCP) and antibody dependent cell-mediated cytotoxicity (ADCC), the two main mechanisms by which immune effector cells can kill tumour cells. Due to its rapid binding to Fcε-receptors on cells, IgE is quickly removed from the circulation and has a significantly longer tissue half-life than IgG (2 weeks versus 2-3 days), which is advantageous in terms of side-effects because of the short duration of the compound in the bloodstream and also supports a role in the killing of solid tumours.

Moreover, potential IgE-immunotherapies should be effectively distributed to tumour tissues because IgE antibodies bound to Fee-receptors on e.g. mast cells can use those cells as shuttle systems to penetrate malignancies and, because mast cells are tissue-resident immune cells (St John A. L., Abraham S. N. (2013) J. Immunol. 190: 4458-4463), this transport would be highly efficient.

Other possible advantages include the high sensitivity of IgE-effector cells to activation by antigens and the speed and amplitude of the response, which can be seen most impressively during allergic and anaphylactic reactions, typically beginning within minutes upon allergen exposure. At the same time this is also the biggest concern of using IgE-based immunotherapies against cancer: recombinant IgE, applied intravenously, always bears the risk of anaphylactic reactions. Therefore, careful selection of the target epitope is of uttermost importance in this regard.

Accordingly, there is a need for antibodies having improved properties compared to both IgE and IgG isotypes, and that are useful for example in the treatment of cancer.

### SUMMARY OF THE INVENTION

Despite the advantages of IgE over IgG in the solid tumour setting, IgG possesses certain functions that IgE lacks, such as a longer half-life compared to IgE. Therefore, by exploiting the high degree of structural similarity among immunoglobulin domains, the present invention provides in one aspect IgE/IgG hybrid antibodies that possess the combined functionality of the IgG and IgE isotypes.

In one aspect, the present invention provides a hybrid antibody that binds FCC receptors and neonatal Fc receptor (FcRn). In this context, “binds” typically refers to binding of the hybrid antibody via one or more constant domains thereof, i.e. “binds” does not refer to specificity of the hybrid antibody binding to target antigen via its variable domains. Preferably the hybrid antibody binds to FcRn in a pH-dependent manner. For instance, the hybrid antibody may have a higher affinity for FcRn at pH 6.0 than at pH 7.4.

The term hybrid refers herein to an antibody whose structure is derived from more than one class of antibody. In the present invention, it is typically the Fc region that is a hybrid, thereby providing the antibody with the capability to bind to cell surface receptors of the immune system that are associated with different classes of antibody. Typically, the hybrid antibody is capable of binding to and activating both an FCC receptor and a FcRn receptor, thereby transducing receptor signalling and effector functions in cells of immune system in which these receptors are expressed.

In one embodiment, the antibody of the present invention comprises one or more heavy chain constant domains derived from an IgE antibody (e.g. derived from an c heavy chain). For instance, the antibody may comprise one or more domains selected from Cε1, Cε2, Cε3 and Cε4. Preferably the antibody comprises at least a Cε3 domain, more preferably at least Cε2, Cε3 and Cε4 domains.

In one embodiment, the hybrid antibody may comprise a tetrameric IgE having an Fc region comprising CH2, CH3 and CH4 domains derived from IgE (i.e. Cε2, Cε3 and Cε4 domains) in which one or more of the constant domains may include one or more amino acid substitutions that are identified as being pertinent to FcRn binding in IgG. FcRn binding may be provided by one or more amino acid substitutions in at least one Fc domain of the tetrameric IgE. The fragment crystallisable/constant region (Fc region) is the tail region of an antibody that interacts with cell surface Fc receptors and some proteins of the complement system. This property allows antibodies to activate the immune system.

The amino acid substitution may be made in either or both of Cε3 and Cε4 of IgE. The substitution may be replacement of a native residue in IgE with an amino acid found at a corresponding position in IgG, so that the FcRn binding property of IgG may be imparted into IgE. For example, the Cε3Cε4 domain of IgE may include one or more His substitutions, thereby enabling FcRn binding by IgE (e.g in a pH-dependent manner). The tetrameric IgE may comprise a Fab region and an Fc region where the Fc domain comprises at least Cε2, Cε3 and Cε4 domains.

In another embodiment, the hybrid antibody comprises a tetrameric IgE having an Fc region comprising CH2, CH3 and CH4 domains derived from IgE (i.e. Cε2, Cε3 and Cε4 domains) in which one or more of the constant domains may include all or part of a binding site for FcRn derived from an IgG antibody. A FcRn receptor binding site or sequence may be provided by way of one or more sequences derived from IgG found in one or more constant domains of IgG. Structural regions on IgE that exhibit homology to the regions on IgG where FcRn binds may be identified. Having identified such regions, amino acid and/or sequence substitutions may then be made to enable transfer of IgG functionality onto an IgE background.

Thus in one embodiment, the hybrid antibody comprises an IgE Cε3 domain comprising a histidine residue at position 78. For instance the hybrid antibody may comprise a IgE CH3 domain as defined in SEQ ID NO:2, or a variant or fragment thereof, comprising the mutation T78H. In this context, the numbering refers to the amino acid residue position from the start of the IgE Cε3 domain, i.e. the amino acid residue at the N-terminus of the IgE Cε3 domain is position 1. Variants and fragments of SEQ ID NO:2 include sequences having at least 85%, 90%, 95% or 99% sequence identity with the sequence of SEQ ID NO:2, e.g. over at least 30, 50 or 100 amino acid residues of, or over the the full length of SEQ ID NO:2 and fragments of a similar length, provided that the sequence retains the functional properties of an antibody comprising SEQ ID NO:2 and comprising the mutation T78H, e.g. binding to an Fcc receptor and FcRn.

In another embodiment, the hybrid antibody comprises an IgE Cε4 domain comprising a histidine residue at position 95. For instance, the hybrid antibody may comprise an IgE CH4 domain as defined in SEQ ID NO:3, or a variant or fragment thereof, comprising the mutation S95H. In another embodiment, the hybrid antibody comprises an IgE Cε4 domain comprising a histidine residue at position 98. For instance, the hybrid antibody may comprise an IgE CH4 domain as defined in SEQ ID NO:3, or a variant or fragment thereof, comprising the mutation Q98H. In this context, the numbering refers to the amino acid residue position from the start of the IgE Cε4 domain, i.e. the amino acid residue at the N-terminus of the IgE Cε4 domain is position 1. Variants and fragments of SEQ ID NO:3 include sequences having at least 85%, 90%, 95% or 99% sequence identity with the sequence of SEQ ID NO:3, e.g. over at least 30, 50 or 100 amino acid residues of, or over the the full length of SEQ ID NO:3 and fragments of a similar length, provided that the sequence retains the functional properties of an antibody comprising SEQ ID NO:3 and comprising the mutation S95H and/or Q98H, e.g. binding to an Fcε receptor and FcRn. Preferably the hybrid antibody comprises 2 or 3 histidine substitutions, e.g. the antibody comprises an IgE Cε3 domain comprising a histidine residue at position 78 and/or an IgE Cε4 domain comprising a histidine residue at position 95 and/or 98. In a particularly preferred embodiment, the hybrid antibody comprises an IgE CH3 domain as defined in SEQ ID NO:2, or a variant or fragment thereof, comprising the mutation T78H and/or an IgE CH4 domain as defined in SEQ ID NO:3, or a variant or fragment thereof, comprising the mutation S95H and/or Q98H.

Thus in further preferred embodiments, the hybrid antibody may comprise an IgE Cε3 loop sequence as defined in SEQ ID NO:31 (i.e. PVGHR) and/or an IgE Cε4 loop sequence as defined in SEQ ID NO:32 or 33 (i.e. AHPSHTV or RAVHEAAHPSHTV).

Alternatively, the FcRn receptor binding site may be attached to the C-terminal of IgE, for example by way of one or more Fcγ domains derived from IgG. Expressed in another way, the hybrid antibody may comprise an Fc region comprising CH2, CH3 and CH4 domains derived from IgE (i.e. Cε2, Cε3 and Cε4 domains), and a CH2 domain, or variant thereof, derived from IgG (i.e. a Cγ2 domain). The antibody may further comprise the CH3 domain, or variant thereof, derived from IgG (i.e. a Cγ3 domain) and/or all or part of the hinge region derived from IgG.

Attachment of the one or more constant domains may be by any suitable attachment, link, graft, fixation or fusion. For example, the construct may include all or part of the hinge region derived from IgG. It will be appreciated that all or part of the constant domain sequence may be used, as well as variants thereof.

The antibody domains described herein may be derived from any species, preferably a mammalian species, more preferably from human.

In one embodiment, the hybrid antibody binds to FcRn and FcεRI.

It will be appreciated that other receptor binding sites and desirable functions specific to IgG in the context of tumour targeting may also be grafted onto or into an IgE molecule to alter its functionality.

The hybrid antibody may further comprise a variable domain sequence that determines specific binding to one or more target antigen(s). Such variable domain sequences may be derived from any immunoglobulin isotype (e.g. IgA, IgD, IgE, IgG or IgM). In one embodiment, the variable domain sequence may be derived from IgE. In another embodiment, the variable domain sequence may be derived from IgG, e.g. IgG1. Alternatively, the variable domains may comprise sequences derived from two or more different isotypes, e.g. the variable domain may comprise a partial sequence derived from IgE and a partial sequence derived from IgG1. In one embodiment, the hybrid antibody comprises one or more complementarity-determining regions (CDRs) derived from an immunoglobulin isotype other than IgE (e.g. IgA, IgD, IgG or IgM, for example IgG1), and one or more framework regions and/or constant domains derived from an immunoglobulin of the isotype IgE.

The variable domains or portions thereof (e.g. the complementarity-determining regions (CDRs) or framework regions) may also be derived from the same or a different mammalian species to the constant domains present in the hybrid antibody. Thus, the hybrid antibody may be a chimaeric antibody, a humanised antibody or a human antibody.

Typically the variable domain(s) of the antibody binds to one or target antigens useful in the treatment of cancer, e.g. to a cancer antigen (i.e. an antigen expressed selectively on cancer cells or overexpressed on cancer cells) or to an antigen that inhibits or suppresses immune-mediated tumour cell killing. A sequence of one such variable domain sequence (i.e. of trastuzumab (Herceptin) IgE that binds to the cancer antigen HER2/neu) is shown in SEQ ID NO:1.

In one embodiment, the antibody may comprise an IgE amino acid sequence as defined in SEQ ID NO: 26. For instance, the hybrid antibody may comprise an amino acid sequence having at least 85%, 90%, 95% or 99% sequence identity with the sequence of SEQ ID NO:26, e.g. over at least 50, 100 or 200 amino acid residues of, or over the the full length of SEQ ID NO:26. Preferably the antibody comprises at least one, two or three histidine substitutions with respect to a wild type IgE CH3 and/or CH4 sequence, e.g. the hybrid antibody comprises a histidine residue at position(s) 78, 203 and/or 206 of SEQ ID NO:26.

In another embodiment, the antibody may comprise an IgE (e.g. heavy chain) amino acid sequence as defined in SEQ ID NO: 34. For instance, the hybrid antibody may comprise an amino acid sequence having at least 85%, 90%, 95% or 99% sequence identity with the sequence of SEQ ID NO:34, e.g. over at least 50, 100, 200, 300 or 500 amino acid residues of, or over the the full length of SEQ ID NO:34. Preferably the antibody comprises at least one, two or three histidine substitutions with respect to a wild type IgE CH3 and/or CH4 sequence, e.g. the hybrid antibody comprises a histidine residue at position(s) 408, 533 and/or 536 of SEQ ID NO:34. In these embodiments, the antibody preferably further comprises a light chain amino acid sequence as defined in SEQ ID NO: 35, or an amino acid sequence having at least 85%, 90%, 95% or 99% sequence identity with the sequence of SEQ ID NO:35, e.g. over at least 50, 100, 200, 300 or 500 amino acid residues of, or over the the full length of SEQ ID NO:35.

In another embodiment, the antibody may comprise an IgE (e.g. heavy chain) amino acid sequence as defined in SEQ ID NO: 186. For instance, the hybrid antibody may comprise an amino acid sequence having at least 85%, 90%, 95% or 99% sequence identity with the sequence of SEQ ID NO:186, e.g. over at least 50, 100, 200, 300 or 500 amino acid residues of, or over the the full length of SEQ ID NO:186. Preferably the antibody comprises at least one, two or three histidine substitutions with respect to a wild type IgE CH3 and/or CH4 sequence, e.g. the hybrid antibody comprises a histidine residue at position(s) 411, 536 and/or 539 of SEQ ID NO:186. In these embodiments, the antibody preferably further comprises a light chain amino acid sequence as defined in SEQ ID NO: 187 or 189, or an amino acid sequence having at least 85%, 90%, 95% or 99% sequence identity with the sequence of SEQ ID NO: 187 or 189, e.g. over at least 50, 100, 200, 300 or 500 amino acid residues of, or over the the full length of SEQ ID NO: 187 or 189.

In another embodiment, the antibody may comprise an IgE (e.g. heavy chain) amino acid sequence as defined in SEQ ID NO: 188. For instance, the hybrid antibody may comprise an amino acid sequence having at least 85%, 90%, 95% or 99% sequence identity with the sequence of SEQ ID NO:188, e.g. over at least 50, 100, 200, 300 or 500 amino acid residues of, or over the the full length of SEQ ID NO:188. Preferably the antibody comprises at least one, two or three histidine substitutions with respect to a wild type IgE CH3 and/or CH4 sequence, e.g. the hybrid antibody comprises a histidine residue at position(s) 410, 535 and/or 538 of SEQ ID NO:188. In these embodiments, the antibody preferably further comprises a light chain amino acid sequence as defined in SEQ ID NO: 187 or 189, or an amino acid sequence having at least 85%, 90%, 95% or 99% sequence identity with the sequence of SEQ ID NO: 187 or 189, e.g. over at least 50, 100, 200, 300 or 500 amino acid residues of, or over the the full length of SEQ ID NO: 187 or 189.

In some embodiments, the antibody may comprise an IgE amino acid sequence as defined in any one or more of SEQ ID NOs: 15 to 25, or a variant or fragment thereof. For instance, the hybrid antibody may comprise an amino acid sequence having at least 85%, 90%, 95% or 99% sequence identity with any one or more of the sequences of SEQ ID NOs:15 to 25.

In another embodiment, the hybrid antibody comprises an IgG CH2 amino acid sequence having at least 85%, 90%, 95% or 99% sequence identity with SEQ ID NO:9. In another embodiment, the antibody further comprises an IgG CH3 amino acid sequence having at least 85%, 90%, 95% or 99% sequence identity with SEQ ID NO:10. In another embodiment, the antibody further comprises an IgG hinge amino acid sequence having at least 85%, 90%, 95% or 99% sequence with SEQ ID NO:8.

In a particular embodiment, the antibody comprises: i) an (e.g. IgE-derived) amino acid sequence having at least 85%, 90%, 95% or 99% sequence identity with SEQ ID NO:1 to 3, preferably an amino acid sequence having at least 85%, 90%, 95% or 99% sequence identity with each of SEQ ID NO:1, SEQ ID NO:2 and SEQ ID NO:3; and ii) an (e.g. IgG-derived) amino acid sequence having at least 85%, 90%, 95% or 99% sequence identity with SEQ ID NO:8, 9 and/or 10 (more preferably at least SEQ ID NO:9 and SEQ ID NO:10).

The IgG-derived amino acid sequence is preferably attached to the C terminal of the IgE-derived amino acid sequence, either directly or using a suitable linker sequence. For instance, the sequence of SEQ ID NO:3 may be adjacent to the sequence of SEQ ID NO:8, 9 or 10, preferably SEQ ID NO:8. Thus in some embodiments, the hybrid antibody may comprise at least a Cε4 domain and at least an IgG hinge region and Cγ2 domains, preferably at least a Cε4 domain and at least an IgG hinge region and Cγ2 and Cγ3 domains. Thus, the antibody may comprise an amino acid sequence having at least 85%, 90%, 95% or 99% sequence identity with SEQ ID NO:27 or SEQ ID NO:28.

In preferred embodiments, the antibody comprises a (e.g. heavy chain) amino acid sequence having at least 85%, 90%, 95% or 99% sequence identity with SEQ ID NO:29 or SEQ ID NO:30, most preferably SEQ ID NO:30, for example over at least 50, 100, 200, 300, 500 or 700 amino acid residues of, or over the full length of, SEQ ID NO:29 or SEQ ID NO:30.

Also described herein are antibodies comprising at least a CH3 domain or fragment thereof derived from IgE (i.e. a Cε3 domain) and one or more loop sequences derived from an IgG CH2 domain (i.e. a Cγ2 domain). Such antibodies may comprise a Cε3 domain in which one or more loop sequences (e.g. as defined in SEQ ID NOs: 4 and 5) are replaced by one or more FcRn-binding loops derived from a Cγ2 domain (e.g. as defined in SEQ ID NOs: 11 and 12). The loop sequences that are replaced in the Cε3 domain of IgE may show structural homology to the FcRn-binding loops in the Cγ2 domain of IgG. Such antibodies may comprise an amino acid sequence (e.g. encoding a hybrid Cε3/Cγ2 domain) having at least 85%, 90%, 95% or 99% sequence identity with any one or more of the sequences of SEQ ID NOs:15, 16, 19 to 25.

Also described herein are antibodies comprising at least a CH4 domain or fragment thereof derived from IgE (i.e. a Cε4 domain) and one or more loop sequences derived from an IgG CH3 domain (i.e. a Cγ3 domain). Such antibodies may comprise a Cε4 domain in which one or more loop sequences (e.g. as defined in SEQ ID NOs: 6 and 7) are replaced by one or more FcRn-binding loops derived from a Cγ3 domain (e.g. as defined in SEQ ID NO: 13 and 14). The loop sequences that are replaced in the Cε4 domain of IgE may show structural homology to the FcRn-binding loops in the Cγ3 domain of IgG. Such antibodies may comprise an amino acid sequence (e.g. encoding a hybrid Cε4/Cγ3 domain) having at least 85%, 90%, 95% or 99% sequence identity with any one or more of the sequences of SEQ ID NOs:17, 18 and 20 to 25.

In another aspect the invention encompasses a hybrid antibody as defined hereinabove for use in treating or preventing cancer, e.g. benign or malignant tumours. Expressed in another way, the invention encompasses use of a hybrid antibody as described hereinabove in the manufacture of a medicament for administration to a human or animal for treating, preventing or delaying cancer, e.g. benign or malignant tumours. In another aspect, the invention encompasses a method of preventing, treating and/or delaying cancer (e.g. benign or malignant tumours) in a mammal suffering therefrom, the method comprising administering to the mammal a therapeutically effective amount of the hybrid antibody as described hereinabove.

The cancer may be e.g. melanoma, Merkel cell carcinoma, non-small cell lung cancer (squamous and non-squamous), renal cell cancer, bladder cancer, head and neck squamous cell carcinoma, mesothelioma, virally induced cancers (such as cervical cancer and nasopharyngeal cancer), soft tissue sarcomas, haematological malignancies such as Hodgkin's and non-Hodgkin's disease and diffuse large B-cell lymphoma (for example melanoma, Merkel cell carcinoma, non-small cell lung cancer (squamous and non-squamous), renal cell cancer, bladder cancer, head and neck squamous cell carcinoma and mesothelioma or for example virally induced cancers (such as cervical cancer and nasopharyngeal cancer) and soft tissue sarcomas. It will be appreciated that the hybrid antibody of the invention may be administered in the form of a pharmaceutically acceptable composition or formulation.

In yet another aspect, the present invention resides in a composition comprising a hybrid antibody as described hereinabove and a pharmaceutically acceptable excipient, diluent or carrier. Optionally, the composition may further comprise a therapeutic agent such as another antibody or fragment thereof, aptamer or small molecule. The composition may be in sterile aqueous solution.

In a yet further aspect, there is provided a (recombinant) nucleic acid that encodes all or part of a heavy chain of a hybrid antibody, wherein the heavy chain comprises an amino acid sequence having at least 85%, 90%, 95% or 99% sequence identity with (i) SEQ ID NO:1, and (ii) any one or more of SEQ ID NOs:15 to 26, preferably SEQ ID NO:26.

In a further aspect, there is provided a (recombinant) nucleic acid that encodes all or part of a heavy chain of a hybrid antibody, wherein the heavy chain comprises an amino acid sequence having at least 85%, 90%, 95% or 99% sequence identity with SEQ ID NO:34.

In a yet further aspect, there is provided a (recombinant) nucleic acid that encodes all or part of a heavy chain of a hybrid antibody, wherein the heavy chain comprises an amino acid sequence having at least 85%, 90%, 95% or 99% sequence identity with (i) one or more of SEQ ID NO:1, 2 and 3, and (ii) SEQ ID NOs:8 and SEQ ID NOs:9 and/or SEQ ID NO:10. In one embodiment, the nucleic acid encodes an amino acid sequence having at least 85%, 90%, 95% or 99% sequence identity with SEQ ID NO:9 or SEQ ID NO:30.

There is also provided a vector comprising the nucleic acid as defined above, optionally wherein the vector is a CHO vector (i.e. an expression vector suitable for expression of the hybrid antibody in Chinese Hamster Ovary (CHO) cells).

In a further aspect, there is provided a host cell comprising a recombinant nucleic acid encoding a hybrid antibody as described hereinabove or a vector as described herein, wherein the encoding nucleic acid is operably linked to a promoter suitable for expression in mammalian cells.

Also provided herein is a method of producing the hybrid antibody described hereinabove comprising culturing host cells as described herein under conditions for expression of the antibody and recovering the antibody or a fragment thereof from the host cell culture.

### BRIEF DESCRIPTION OF THE DRAWINGS

FIG. 1: A schematic diagram of single cycle kinetic analysis of IgE variant antibodies binding to FcRn.

FIG. 2: Assay results showing binding of hybrid antibodies to FcRn.

FIG. 3: Assay results showing binding of IgE variant antibodies and fusion constructs to FcRn using biotin capture at pH 6.0.

FIG. 4: A schematic diagram of multiple cycle kinetic analysis of IgE variant antibodies binding to FcRn.

FIG. 5: An illustration of steady state analysis showing the conversion of raw data to a sensorgram.

FIG. 6: Assay results showing binding of IgG1, IgG4 and IgE_IgG_CH2_CH3 fusion protein to FcRn using FcRn capture at pH 6.0.

FIG. 7: Assay results showing binding of Herceptin, wild-type IgE, IgE_IgG_CH2_CH3, IgE containing 3× IgG Histadine residues, IgE containing IgG FcRn Loop 2 and Loop 3a, IgE containing IgG FcRn Loop 1 and IgE containing IgG FcRn Loop 1, Loop 2 and Loop 3a to human FcRn at pH 6.0.

FIG. 8: Assay results showing binding of IgG1, IgG4 and IgE_IgG_CH2 _CH3 fusion protein to FcRn using FcRn capture at pH 7.4.

FIG. 9: Assay results showing binding of Herceptin, wild-type IgE, IgE_IgG_CH2_CH3, IgE containing 3× IgG Histadine residues, IgE containing IgG FcRn Loop 2 and Loop 3a, IgE containing IgG FcRn Loop 1 and IgE containing IgG FcRn Loop 1, Loop 2 and Loop 3a to human FcRn at pH 7.4.

FIG. 10: Schematic of the vector expressing the IGEG.

FIG. 11: Schematic of the Biacore assay used to assess the binding of the Trastuzumab IGEG variants to human Her2 antigen by single cycle kinetic analysis.

FIG. 12: Human HER2: 1:1 binding of Trastuzumab IGEG variants. Constructs are as described in Example 5.

FIG. 13: Schematic of the Biacore assay used to assess antibody binding to Fc gamma receptors.

FIG. 14: HMW-MAA IGEG (CH) variant binding to human Fc receptors. (a) Human FcgRI: 1:1 binding of HMW-MAA-IGEG variants. (b) Human Fce RIa: 1:1 binding of HMW-MAA IGEG variants. (c) Human FcγRIIIA_{176Val}: Binding of HMW-MAA IGEG variants—Raw Sensorgrams. (d) Human FcγRIIIA_{176Val}: Steady State binding of HMW-MAA IGEG variants—Analysed Data. In this figure, “CH” refers to anti-HMW-MAA (i.e. CSPG4), the variant designations are otherwise as described in Example 5.

FIG. 15: Schematic of the Biacore assay used to assess antibody binding to FcRn.

FIG. 16: HMW-MAA (CH) IGEG variant binding to human FcRn (a) FcRn pH 6.0: Binding of HMW-MAA IGEG variants—Raw Sensorgrams. (b) FcRn pH 6.0: Steady State binding of HMW-MAA IGEG variants—Analysed Data. (c) FcRn pH 7.4: Binding of HMW-MAA IGEG variants—Raw Sensorgrams (d) FcRn pH 7.4: Steady State binding of HMW-MAA IGEG variants—Analysed Data. In this figure, “CH” refers to anti-HMW-MAA (i.e. CSPG4), the variant designations are otherwise as described in Example 5.

FIG. 17: Biostability analysis of HMW-MAA (Hu CH) IGEG variants. (a) Fluorescence Thermal Melting Curves Overlay. (b) SLS 473 Stability Profile Curves Overlay. In this figure, “CH” refers to anti-HMW-MAA (i.e. CSPG4), the variant designations are otherwise as described in Example 6.

FIG. 18. Binding of anti-HMW-MAA (HuCH) IGEG Antibodies to A375 cells (a) Detection with anti-IgG secondary Antibody. (b) Detection with anti-IgE secondary Antibody. In this figure, “CH” refers to anti-HMW-MAA (i.e. CSPG4), the variant designations are otherwise as described in Examples 4 and 5. huCH IgE 3-His refers to an antibody as described in

Example 4, e.g. comprising heavy and light chain sequences as defined in SEQ ID NOs: 188 and 189.

FIG. 19: R1, R2, R3 gating of data acquired from the Attune™ N×T Acoustic Focusing Cytometer.

FIG. 20: Effects of the Trastuzumab IgG, Herceptin IgG, Trastuzumab-IGEG (labelled CH2CH3), Trastuzumab-IGEG-C220S (labelled CH2CH3C220S) and Isotype IgG antibodies on antibody-dependent cell-mediated phagocytosis (ADCP) and antibody-dependent cell-mediated cytotoxicity (ADCC). (a) The effects of the antibodies on ADCP and ADCC at different concentrations (120-7.5nM). (b) Graph showing the effects of the antibodies on ADCP and ADCC.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6, or ≥7 etc. of said members, and up to all said members.

As used herein, the term “antibody” is used in its broadest sense and generally refers to an immunologic binding agent. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

An antibody may be a polyclonal antibody, e.g., an antiserum or immunoglobulins purified there from (e.g., affinity-purified). An antibody may be a monoclonal antibody or a mixture of monoclonal antibodies. Monoclonal antibodies can target a particular antigen or a particular epitope within an antigen with greater selectivity and reproducibility. By means of example and not limitation, monoclonal antibodies may be made by the hybridoma method first described by Kohler et al 1975 (Nature 256: 495) or may be made by recombinant DNA methods (e.g., as in U.S. patent application Ser. No. 4,816,567). Monoclonal antibodies may also be isolated from phage antibody libraries using techniques as described by Clackson et al 1991 (Nature 352: 624-628) and Marks et al 1991 (J. Mol. Biol. 222: 581-597), for example.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse.

A skilled person will understand that an antibody may include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

Hence, also disclosed are methods for immunising animals, e.g., non-human animals such as laboratory or farm, animals using (i.e., using as the immunising antigen) any one or more (isolated) markers, peptides, polypeptides or proteins and fragments thereof as taught herein, optionally attached to a presenting carrier. Immunisation and preparation of antibody reagents from immune sera is well-known per se and described in documents referred to elsewhere in this specification. The animals to be immunised may include any animal species, preferably warm-blooded species, more preferably vertebrate species, including, e.g., birds, fish, and mammals. Without limitation, the antibodies may be chicken, turkey, goose, duck, guinea fowl, shark, quail or pheasant. Also without limitation, the antibodies may be human, murine (e.g., mouse, rat, etc.), donkey, rabbit, goat, sheep, guinea pig, shark, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

The invention described herein resides in IgE antibodies with an engineered heavy chain (Fc) portion resulting in hybrid IgE molecules. Structural regions of the CH3 and CH4 domains of IgE were identified that exhibited homology to similar regions on IgG where FcRn binds. Having identified such regions, amino acid substitutions were made that enabled transfer of IgG functionality onto an IgE background. In particular, amino acids or sequences in one or more loops in one or more constant domains of IgE were replaced with IgG FcRn amino acids or sequences to impart FcRn functionality into IgE.

The hybrid antibodies described herein are typically capable of binding to Fcε receptors, e.g. to the FcεRI and/or the FcεRII receptors. Preferably the antibody is at least capable of binding to FcεRI (i.e. the high affinity Fεe receptor) or is at least capable of binding to FcεRII (CD23, the low affinity Fcε receptor).

Typically, the antibodies are also capable of activating Fcε receptors, e.g. expressed on cells of the immune system, in order to initiate effector functions mediated by IgE. For instance, the antibodies may be capable of binding to FcεRI and activating mast cells, basophils, monocytes/macrophages and/or eosinophils.

The sites on IgE responsible for these receptor interactions have been mapped to peptide sequences on the Cε chain and are distinct. The FcεRI site lies in a cleft created by residues between Gln 301 and Arg 376 and includes the junction between the Ca and Cε3 domains (Helm, B. et al. (1988) Nature 331, 180183). The FcεRII binding site is located within Cε3 around residue Val 370 (Vercelli, D. et al. (1989) Nature 338, 649-651). A major difference distinguishing the two receptors is that FcεRI binds monomeric Cε, whereas FcεRII will only bind dimerised Cε, i.e. the two Cε chains must be associated. Although IgE is glycosylated in vivo, this is not necessary for its binding to FcεRI and FcεRRII. Binding is in fact marginally stronger in the absence of glycosylation (Vercelli, D. et al (1989) supra).

Thus, binding to Fcc receptors and related effector functions are typically mediated by the heavy chain constant domains of the antibody, in particular by domains which together form the Fc region of the antibody. The antibodies described herein typically comprise at least a portion of an IgE antibody e.g. one or more constant domains derived from an IgE, preferably a human IgE. In particular embodiments, the antibodies comprise one or more domains (derived from IgE) selected from Cε1, Cε2, Cε3 and Cε4. In one embodiment, the antibody comprises at least Cε2 and Cε3, more preferably at least Cε2, Cε3 and Cε4, preferably wherein the domains are derived from a human IgE. In one embodiment, the antibody comprises an epsilon (ε) heavy chain, preferably a human ε heavy chain.

Constant domains derived from human IgE, in particular Cε1, Cε2, Cε3 and Cε4 domains, are shown in SEQ ID NOs: 1, 2 and 3 respectively. Nucleic acid sequences encoding these acid sequences may be deduced by a skilled person according to the genetic code. The amino acid sequences of other human and mammalian IgEs and domains thereof, including human Cε1, Cε2, Cε3 and Cε4 domains and human c heavy chain sequences, are known in the art and are available from public-accessible databases. For instance, databases of human immunoglobulin sequences are accessible from the International ImMunoGeneTics Information System (IMGT®) website at http://www.imgt.org. As one example, the sequences of various human IgE heavy (ε) chain alleles and their individual constant domains (Cε1-4) are accessible at http://www.imgt.org/IMGT_GENE-DB/GENElect? query=2+IGHE&species=Homo+sapiens.

The hybrid antibodies described herein are typically capable of further binding to the foetal Fc (FcRn) receptor. Preferably the hybrid antibodies are capable of binding to and activating FcRn and/or activating cells of the immune system expressing such receptors (including myeloid cells of the haematopoietic system such as e.g. monocytes, macrophages, neutrophils, basophils and eosinophils).

Preferably the hybrid antibodies bind to FcRn in a pH-dependent manner. In particular, the hybrid antibody may preferentially bind to FcRn at an acidic pH, e.g. the antibody may have a higher affinity for FcRn at a pH below 7 compared to at pH 7 or above. For instance, in one embodiment the antibody binds to FcRn at a pH of 4 to 6.5 (e.g. at pH 6.0) but not at pH 7.0 or 7.4.

The antibodies described herein typically comprise at least a portion of an IgG antibody that is responsible for the binding of IgG to FcRn, e.g. one or more sequences or amino acid substitutions derived from an IgG (e.g. an IgG1), preferably a human IgG. In a particular embodiment, the antibodies comprise one or more amino acid substitutions in at least one Fc domain of a tetrameric IgE. For example, at least one amino acid substitution may be made in Cε3 of IgE. Alternatively or in addition, at least one amino acid substitution may be made in Cε4 of IgE. Specifically, one amino acid substitution may be made in Cε3 and two amino acid substitutions may be made in Cε4 of IgE.

Preferably at least one native amino acid present in IgE, e.g. in a Cε3 or Cε4 domain of IgE, is substituted for histidine. Thus the hybrid antibody may be an IgE comprising one or more non-native histidine residues, i.e. residues that are not typically histidine at that position in an IgE sequence. Typically the non-native histidine residues are present at a position in the IgE antibody corresponding to a position in an IgG antibody at which a histidine residue is present. Thus the IgE antibody typically comprises one, two or three heterologous histidine residues, that may confer FcRn binding to the IgE antibody. In this context “heterologous” or “non-native” means derived from a genotypically distinct entity from that of the rest of the entity to which it is being compared. For example, an amino acid residue or sequence derived from a particular protein or polypeptide that is introduced by genetic engineering techniques into a different polypeptide is a heterologous or non-native residue. Thus, for example, an IgE antibody that includes a histidine residue at a position that is not normally histidine in a naturally-occurring, wild-type or native IgE domain is said to comprise a heterologous or non-native histidine residue at that position.

For example, a threonine residue may be substituted for histidine in Loop 2 of Cε3 of IgE. Additionally or alternatively, a serine residue may be substituted for histidine and glutamine may be substituted for histidine in Loop 3 of Cε4 of IgE. Examples of such variants may be found in SEQ ID NOS: 26 and 31 to 34.

In another embodiment, the antibodies comprise sequences derived from IgG selected from loop sequences found in Cγ2 and/or Cγ3. In one embodiment, the antibody comprises at least part of a loop sequence derived from Cγ2, more preferably at least Cγ2 and Cγ3, preferably wherein the domains are derived from a human IgG1 antibody. In one embodiment, the antibody further comprises a hinge region derived from IgG, e.g. IgG1.

Constant domains Cγ2 and Cγ3 derived from human IgG are shown in SEQ ID NOs: 9 and 10 respectively. The hinge domain derived from from human IgG is set out in SEQ ID NO:8. Nucleic acid sequences encoding these acid sequences may be deduced by a skilled person according to the genetic code. The amino acid sequences of other human and mammalian IgG constant domains, including human Cγ2 and Cγ3 domains and hinge sequences, are known in the art and are available from public-accessible databases, as described above for IgE constant domains.

The amino acid sequences of one or more IgE domain and one or more IgG domains may be linked directly or via a suitable linker. Suitable linkers for joining polypeptide domains are well known in the art, and may comprise e.g. 1, 2, 3, 4, 5, 6, 7, 8, 9 or 10 amino acid residues. In some embodiments, the linker sequence may comprise up to 20 amino acid residues.

Binding of the hybrid antibodies to Fcε and FcRn receptors may be assessed using standard techniques. Binding may be measured e.g. by determining the antigen/antibody dissociation rate, by a competition radioimmunoassay, by enzyme-linked immunosorbent assay (ELISA), or by Surface Plasmon Resonance (e.g. Biacore). Binding affinity may also be calculated using standard methods, e.g. based on the Scatchard method as described by Frankel et al (1979) Mol. Immunol. 16:101-106.

In general, functional fragments of the sequences defined herein may be used in the present invention. Functional fragments may be of any length (e.g. at least 50, 100, 300 or 500 nucleotides, or at least 50, 100, 200, 300 or 500 amino acids), provided that the fragment retains the required activity when present in the antibody (e.g binding to FcRn and/or a Fcε receptor).

Variants of the amino acid and nucleotide sequences described herein may also be used in the present invention, provided that the resulting antibody binds both FcRn and Fcε receptors. Typically such variants have a high degree of sequence identity with one of the sequences specified herein.

The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of the amino acid or nucleotide sequence will possess a relatively high degree of sequence identity when aligned using standard methods.

Methods of alignment of sequences for comparison are well known in the art. Various programs and alignment algorithms are described in: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237; Higgins and Sharp (1989) CABIOS 5:151; Corpet et al (1988) Nucleic Acids Research 16:10881; and Pearson and Lipman (1988) Proc. Natl. Acad. Sci. U.S.A. 85:2444. Altschul et al (1994) Nature Genet. 6:119 presents a detailed consideration of sequence alignment methods and homology calculations.

The NCBI Basic Local Alignment Search Tool (BLAST) (Altschul et al (1990) J. Mol. Biol. 215:403) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of the specific antibody or a domain thereof described herein (e.g. a VL, VH, CL or CH domain) typically have at least about 75%, for example at least about 80%, 90%, 95%, 96%, 97%, 98% or 99% sequence identity with the original sequence (e.g. a sequence defined herein), for example counted over at least 20, 50, 100, 200 or 500 amino acid residues or over the full length alignment with the amino acid sequence of the antibody or domain thereof using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Typically variants may contain one or more conservative amino acid substitutions compared to the original amino acid or nucleic acid sequence. Conservative substitutions are those substitutions that do not substantially affect or decrease the affinity of an antibody to FcRn and/or Fcε receptors. For example, a human antibody that binds the FcRn and/or Fcε may include up to 1, up to 2, up to 5, up to 10, or up to 15 conservative substitutions compared to the original sequence (e.g. as defined above) and retain specific binding to the FcRn and/or Fcε receptor. The term conservative variation also includes the use of a substituted amino acid in place of an unsubstituted parent amino acid, provided that the antibody binds FcRn and/or Fcε. Non-conservative substitutions are those that reduce an activity or binding to FcRn and/or Fcε receptors.

Functionally similar amino acids which may be exchanged by way of conservative substitution are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The domains described above (e.g. one or more IgE and IgG constant domains) are typically present in a heavy chain in the antibody. The hybrid antibody may further comprise one or more light chains in addition to one or more heavy chain sequences as described herein. For instance, in one embodiment the hybrid antibody may comprise a light chain sequence as defined in SEQ ID NO:35, or a fragment or variant thereof. Antibodies are typically composed of a heavy and a light chain, each of which has a variable region, termed the variable heavy (VH) region and the variable light (VL) region. Together, the VH region and the VL region are responsible for binding the antigen recognized by the antibody. Typically, a naturally occurring immunoglobulin has heavy (H) chains and light (L) chains interconnected by disulfide bonds. There are two types of light chain, lambda (λ) and kappa (k). Thus the hybrid antibodies typically comprise two heavy chains and two light chains (e.g. joined by disulfide bonds), e.g. based on an IgE antibody comprising an IgG hinge, CH2 and/or CH3 domain fused at the C-terminus of each heavy chain.

The hybrid antibodies described herein may bind specifically (i.e. via their variable domains or the complementarity determining regions (CDRs) thereof) to one or more target antigens useful in treating cancer. For instance, the hybrid antibodies may bind specifically to one or more cancer antigens (i.e. antigens expressed selectively or overexpressed on cancer cells). The novel combination of effector functions transduced via the combined FcεR- and FcRn-binding capability may enhance cytotoxicity, phagocytosis (e.g. ADCC and/or ADCP) and other cancer cell-killing function of immune system cells (e.g. monocytes/macrophages and natural killer cells). For example, the hybrid antibodies may bind specifically e.g. to EGF-R (epidermal growth factor receptor), VEGF (vascular endothelial growth factor) or erbB2 receptor (Her2/neu). One example of an antibody comprising variable domains that bind selectively to Her2/neu is trastuzumab (Herceptin).

In some embodiments, one or more of the variable domains and/or one or more of the CDRs, preferably at least three CDRs, or more preferably all six CDRs may be derived from one or more of the following antibodies: alemtuzumab (SEQ ID NOs:36-41), atezolizumab (SEQ ID NOs:42-47), avelumab (SEQ ID NOs:48-53), bevacizumab (SEQ ID NOs:54-59), blinatumomab, brentuximab, cemiplimab, certolizumab (SEQ ID NOs:60-65), cetuximab (SEQ ID NOs:66-71), denosumab, durvalumab (SEQ ID NOs:72-77), efalizumab (SEQ ID NOs:78-83), iplimumab, nivolumab, obinutuzumab, ofatumumab, omalizumab (SEQ ID NOs:84-89), panitumumab (SEQ ID Nos:90-95), pembrolizumab, pertuzumab (SEQ ID NOs:96-101), rituximab (SEQ ID NOs:102-107), or trastuzumab (SEQ ID NOs:108-113).

In such embodiments, the variable domains of the antibody may comprise one or more of the CDRs, preferably at least three CDRs, or more preferably all six of the CDR sequences from one of the antibodies listed in Table 1.

TABLE 1
Estimated CDR Amino Acid Sequences for Examples of Antibodies used in Cancer Therapy

| Antibody | CDR H1 | CDR H2 | CDR H3 | CDR L1 | CDR L2 | CDR L3 | Notes |
|---|---|---|---|---|---|---|---|
| Alemtuzumab | GFTF....TDFY | IRDKAKGYTT | AREGHT....AAP | QNIDKY | NT.......N | LQHIS....RPRT | 1A |
| | (36) | (37) | FDY (38) | (39) | (40) | (41) | |
| Atezolizumab | DSWIH | WISPYGGSTY | RHWPG.....GF | DVST.AVA | SASFLY | QQYL.YHPAT | 2B |
| | (42) | (43) | (44) | (45) | (46) | (47) | |
| Avelumab | SYIMM | SIYPSGGITF | .IKLFT..VTTV | VGGYNYVS | DVSNRP | SSYTSSSTRV | 2B |
| | (48) | (49) | (50) | (51) | (52) | (53) | |
| Bevacizumab | GYTF....TNYG | INTY..TGEP | AKYPHYYGSS | QDISNY | FTS | QQYSTVPWT | 1A |
| | (54) | (55) | HWYFDV (56) | (57) | (58) | (59) | |
| Certolizumab | GYVFT.DYGMN | GWL.NTYIGEPI | AR..G.YRSYAM | KASQNVGTN | SASFLY | QQYNIYPL | 3A |
| | (60) | YADSVK.G (61) | DY (62) | VA(63) | (64) | (65) | |
| Cetuximab | GFSL....TNYG | IWSG...GNT | ARALTYY...DY | QSIGTN | YA.......S | QQNNN....WPTT | 1A |
| | (66) | (67) | EFAY (68) | (69) | (70) | (71) | |
| Durvalumab | RYWMS | NIKQDGSEKY | EGGWFG..ELAF | RVSSSYLA | DASSRA | QQYG.SLPWT | 2B |
| | (72) | (73) | (74) | (75) | (76) | (77) | |
| Efalizumab | GYSFT.GHWMN | GIMIHPSDSETR | ARIGIYFYGTT | RASKTISKYL | SGSTLQ | QQHNEYPL | 3A |
| | (78) | YNQKFKDI | YFDYI | A | (82) | (83) | |
| | | (79) | (80) | (81) | | | |
| Omalizumab | GYSITSGYSWN | ASI..TYDGSTNY | ARGSHYF..GH | RASQSV.DYDGD | AASYLE | QQSHEDPY | 3A |
| | (84) | ADSVK.G (85) | WHFAV (86) | SYMN(87) | (88) | (89) | |
| Panitumumab | GGSVS..SGDYY | IYYS...GNT | VRDRVT.....GA | QDISNY | DA.......S | QHFDH....LPLA | 1A |
| | (90) | (91) | FDI (92) | (93) | (94) | (95) | |
| Pertuzumab | GFTF....TDYT | VNPN..SGGS | ARNLGP....SFY | QDVSIG | SAS | QQYYI....YPYT | 1A |
| | (96) | (97) | FDY (98) | (99) | (100) | (101) | |
| Rituximab | GYTF....TSYN | IYPG..NGDT | ARSTYYG..GD | SSVSY | AT.......S | QQWTS....NPPT | 1A |
| | (102) | (103) | WFNV (104) | (105) | (106) | (107) | |
| Trastuzumab | GFNI....KDTY | IYPT..NGYT | SRWGGDG...FY | QDVNTA | SA.......S | QQHYT....TPPT | 1A |
| | (108) | (109) | AMDY (110) | (111) | (112) | (H3) | |

Numbers indicated in brackets are the corresponding SEQ ID NOs.
Dots indicate sequence alignment gaps according to the IMGT and Kabata numbering systems.
Letters indicate the method used to predict the CDR sequence.
A - IMGT, B - Kabat.
1 - Magdelaine-Beuzelin et al. (2007) Structure-function relationships of the variable domains of monoclonal antibodies approved for cancer treatment. Critical Reviews in Oncology/Hematology, 64: 210-225.
2 - Lee et al. (2017). Molecular mechanism ofPD-1/PD-L1 blockade via anti-PD-L1 antibodies atezolizumab and durvalumab. Scientific Reports, 7: 5532.
3 - Ling et al. (2018) Effect of VH-VL Families in Pertuzumab and Trastuzumab Recombinant Production, Her2 and FcγIIA Binding. Frontiers in Immunology, 9: 469.

In alternative embodiments, one or more of the variable domains and/or one or more CDRs, preferably at least three CDRs, or more preferably all six CDRs, may be derived from one or more of the following antibodies: abciximab, adalimumab (SEQ ID NOs:114-119), aducanumab, aducanumab, alefacept, alirocumab, anifrolumab, balstilimab, basiliximab (SEQ ID NOs:120-125), belimumab (SEQ ID NOs:126-131), benralizumab, bezlotoxumab, brodalumab, brolucizumab, burosumab, cankinumab, caplacizumab, crizanlizumab, daclizumab (SEQ ID NOs:132-137), daratumumab, dinutuximab, dostarlimab, duplilumab, eclizumab, elotuzumab, emapalumab, emicizumab, epitinezumab, erenumab, etrolizumab, evinacumab, evolocumab, fremanezumab, galcanezumab, golimumab, guselkumab, ibalizumab, idarucizumab, inebilizumab, infliximab (SEQ ID NOs:138-143), isatuximab, ixekizumab, lanadelumab, leronlimab, margetuximab, mepolizumab, mogamulizumab, muromonab, narsoplimab, natalizumab (SEQ ID NOs:144-149), naxitamab, necitumumab, obiltoxaximab, ocrelizumab, omburtamab, palivizumab (SEQ ID NOs:150-155), ramucirumab, ranibizumab (SEQ ID NOs:156-161), reslizumab, risankizumab, romosozumab, sarilumab, satralizumab, secukinumab, spartalizumab, sutimlimab, tafasitamab, tanezumab, teplizumab, teprotumumab, tildrakizumab, toclizumab, toropalimab, ustekinumab, vedolizumab or zalifrelimab.

In such embodiments, the variable domains of the antibody may comprise one or more of the CDRs, preferably at least three CDRs, or more preferably all six of the CDR sequences from one of the antibodies listed in Table 2.

TABLE 2
Estimated CDR Amino Acid Sequences for Example Therapeutic Antibodies

| Antibody | CDR H1 | CDR H2 | CDR H3 | CDR L1 | CDR L2 | CDR L3 | Notes |
|---|---|---|---|---|---|---|---|
| Adalimumab | DYAMH | AITWNSGHIDYADSVEG | VSYLSTASSLDY | RASQGIRNYLA | AASTLQS | QRYNRAPYT | 1A |
| | (114) | (115) | (116) | (117) | (118) | (119) | |
| Basiliximab | GYSFTR..YWMH | AIYPGNSD..TSYNQKFEG | DYGYYFDF | SASSSRSYMQ | DTSKLAS | HQRSS..YT | 2 |
| | (120) | (121) | (122) | (123) | (124) | (125) | |
| Belimumab | GGTFNNNAIN | GIIPMFGTAKYSQNFQG | SRDLLLFPHHALSP | QGDSLRSYYAS | GKNNRPS | SSRDSSGNHWV | 3B |
| | (126) | (127) | (128) | (129) | (130) | (131) | |
| Daclizumab | GYTFTS..YRMH | YINPSTGY..TEYNQKFKD | GGGVFDY | SASSSISYMH | TTSNLAS | HQRSTYPLT | 2 |
| | (132) | (133) | (134) | (135) | (136) | (137) | |
| Infliximab | IFSNHW | RSKSINSATH | N...YYGSTY | FVGSSIH | KYASESM | QSHSW | 4 |
| | (138) | (139) | (140) | (141) | (142) | (143) | |
| Natalizumab | GFNIKD..TYIH | RIDPANGY..TKYDPKFQG | EGYYGNYGVYAMDY | KTSQDINKYMA | YTSALQP | LQYDN.LWT | 2 |
| | (144) | (145) | (146) | (147) | (148) | (149) | |
| Palivizumab | GFSLSTSGMSVG | DIWWDDK...KDYNPSLKS | SM....ITNWYFDV | KCQLSVGYMH | DTSKLAS | FQGSGYPFT | 2 |
| | (150) | (151) | (152) | (153) | (154) | (155) | |
| Ranibizumab | GYDFTH..YGMN | WINTYTGE..PTYAADFKR | YPYYYGTSHWFDV | SASQDISNYLN | FTSSLHS | QQYSTVPWT | 2 |
| | (156) | (157) | (158) | (159) | (160) | (161) | |

Numbers indicated in brackets are the corresponding SEQ ID NOs.
Dots indicate sequence alignment gaps according to the IMGT and Kabata numbering systems.
Letters indicate the method used to predict the CDR sequence.
A - IMGT, B - Kabat.
1 - Schroter et al. (2014) A generic approach to engineer antibody pH-switches using combinatorial histidine scanning libraries and yeast display. MAbs, 7(1): 138-151.
2 - Wang et al. (2009). Potential aggregation prone regions in biotherapeutics. A survey of commercial monoclonal antibodies. MAbs, 1(3): 254-267.
3 - WO 2015/173782 A1.
4 - Lim et al. (2018). Structural Biology of the TNFα Antagonists Used in theTreatment of Rheumatoid Arthritis. International Journal of Molecular Sciences, 19(3): pii E768.

In other embodiments, one or more of the variable domains and/or one or more of the CDR sequences, preferably at least three CDRs, or more preferably all six CDRs, may be derived from an anti-HMW-MAA antibody. In one embodiment, one or more of the variable domains and/or one or more of the CDR sequences, preferably at least three CDRs, or more preferably all six CDRs may be derived from the anti-HMW-MAA antibody described in WO 2013/050725 (SEQ ID NOs:168 and 169 for the variable domain and SEQ ID NOs:162-167 for CDRs). HMW-MAA refers to high molecular weight-melanoma associated antigen, also known as chondroitin sulfate proteoglycan 4 (CSPG4) or melanoma chondroitin sulfate proteoglycan (MCSP)—see e.g. Uniprot Q6UVK1.

In such embodiments, the variable domains of the antibody may comprise one or more of the CDR sequences, preferably at least three CDRs, or more preferably all six of the CDR sequences defined in Table 3. In other embodiments, one or more of the variable domains of the antibody comprises one or more of the variable domain sequences listed in Table 3.

TABLE 3
Estimated Variable Domains and CDR Sequences
of an Anti-HMW-MAA Antibody

| | SEQ | |
|---|---|---|
| Region | ID NO. | Amino Acid Sequence |
| CDR H1 | 162 | GFTFSNYW |
| CDR H2 | 163 | IRLKSNNFGR |
| CDR H3 | 164 | TSYGNYVGHYFDH |
| CDR L1 | 165 | QNVDTN |
| CDR L2 | 166 | SAS |
| CDR L3 | 167 | QQYNSYPLT |
| Variable | 168 | EQVKLQQSGGGLVQPGGSMKLSCVV |
| Domain | | SGFTFSNYWMNWVRQSPEKGLEWIA |
| (Heavy | | EIRLKSNNFGRYYAESVKGRFTISR |
| Chain) | | DDSKSSAYLQMINLRAEDTGIYYCT |
| | | SYGNYVGHYFDHWGQGTTVTVSS |
| Variable | 169 | DIELTQSPKFMSTSVCDRVSVTCKA |
| Domain | | SQNVDTNVAWYQQKPGQSPEPLLFS |
| (Light | | ASYRYTGVPDRFTGSGSGTDFTLTI |
| Chain) | | SNVQSEDLAEYFCQQYNSYPLTFGG |
| | | GTKLEIK |
| Alternative | 184 | EVQLVQSGGGLVQPGGSLKLSCAVS |
| Variable | | GFTFSNYWMNWVRQAPGKGLEWVGE |
| Domain | | IRLKSNNFGRYYAESVKGRFTISRD |
| (Heavy | | DSKNTAYLQMNSLKTEDTAVYYCTS |
| Chain) | | YGNYVGHYFDHWGQGTLVTVSS |
| Alternative | 185 | DIQLTQSPSFLSASVGDRVTITCKA |
| Variable | | SQNVDTNVAWYQQKPGKAPKPLLFS |
| Domain | | ASYRYTGVPSRFSGSGSGTDFTLTI |
| (Light | | SSLQPEDFATYFCQQYNSYPLTFGG |
| Chain) | | GTKVEIK |

Compositions are provided herein that include a carrier and one or more hybrid antibodies that bind FcRn and Fcε receptors, or functional fragments thereof. The compositions may be prepared in unit dosage forms for administration to a subject. The amount and timing of administration are at the discretion of the treating physician to achieve the desired purposes. The antibody may be formulated for systemic or local (such as intra-tumour) administration. In one example, the antibody may formulated for parenteral administration, such as intravenous administration.

The compositions for administration may include a solution of the antibody or a functional fragment thereof) dissolved in a pharmaceutically acceptable carrier, such as an aqueous carrier. A variety of aqueous carriers may be used, for example, buffered saline and the like. These solutions are sterile and generally free of undesirable matter. These compositions may be sterilised by conventional, well known sterilisation techniques.

The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical dose of the pharmaceutical composition for intravenous administration includes about 0.1 to 15 mg of antibody per kg body weight of the subject per day. Dosages from 0.1 up to about 100 mg per kg per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

Antibodies may be provided in lyophilised form and rehydrated with sterile water before administration, although they are also provided in sterile solutions of known concentration. The antibody solution may be then added to an infusion bag containing 0.9% sodium chloride, USP, and typically administered at a dosage of from 0.5 to 15 mg/kg of body weight. Antibodies may be administered by slow infusion, rather than in an intravenous push or bolus. In one example, a higher loading dose may be administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

The antibody described herein (or functional fragment thereof) may be administered to slow or inhibit the growth of cells, such as cancer cells. In these applications, a therapeutically effective amount of an antibody may be administered to a subject in an amount sufficient to inhibit growth, replication or metastasis of cancer cells, or to inhibit a sign or a symptom of the cancer.

In some embodiments, the antibodies may be administered to a subject to inhibit or prevent the development of metastasis, or to decrease the size or number of metasases, such as micrometastases, for example micrometastases to the regional lymph nodes (Goto et al (2008) Clin. Cancer Res. 14(11):3401-3407).

A therapeutically effective amount of the antibody will depend upon the severity of the disease and the general state of the patient's health. A therapeutically effective amount of the antibody is that which provides either subjective relief of a symptom(s) or an objectively identifiable improvement as noted by the clinician or other qualified observer. These compositions may be administered in conjunction with another chemotherapeutic agent, either simultaneously or sequentially.

Many chemotherapeutic agents are presently known in the art. In one embodiment, the chemotherapeutic agents may be selected from the group consisting of mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones, e.g. anti-androgens, and anti-angiogenesis agents.

All documents cited in the present specification are hereby incorporated by reference in their entirety. The invention will now be described in more detail by way of the following non-limiting examples.

### EXAMPLES

In the following examples, it has been demonstrated that FcRn-binding may be conferred on an IgE antibody by replacing specific amino acids in the CH3 and CH4 domains of IgE with amino acids found in the FcRn binding site of IgG.

### Example 1

### FcRn Constructs

IgE variants were created in which point mutations were made in loops found in the Cε3 and Cε4 domains of IgE. The mutations replaced the indigenous amino acid with histidine at positions known to be involved in IgG-FcRn interactions. The IgE antibody was based on trastuzumab IgE, e.g. as disclosed in Karagiannis et al (2009) Cancer Immunol. Immunother. 58(6):915-30.

Further variant IgE antibodies were generated in which loops in Cε3 and Cε4 domains of the IgE were replaced with one or more FcRn-binding loops derived from Cγ2 and Cγ3 domains of an IgG antibody. The loops that were replaced in the Cε3 and Cε4 domains of the IgE show structural homology to the FcRn-binding loops in the Cγ2 and Cγ3 domains of IgG.

For comparison, two IgE fusion constructs were created in which i) the hinge and Cγ2 domain derived from IgG was fused to the C terminus of trastuzumab IgE, and ii) the IgG hinge and Cγ2 and Cγ3 domains were fused to the C terminus of trastuzumab IgE.

From structural analysis, three loops were identified as being involved in FcRn binding from IgG CH2 (Cγ2) and CH3 (Cγ3). The structurally equivalent loops in IgE were identified and chosen for replacement with the IgG loops. Three loops were identified, L1, L2 and L3, with Loop 3 contained either a truncated substitution (L3a) or an extended substitution (L3b).

Additionally, three Histidine residues were identified within IgG CH2CH3 as being involved in the interaction with FcRn. The equivalent residues in IgE were identified and replaced by Histidine.

DNA sequences corresponding to both the wild type (WT) IgE constant domain and separately, IgE containing IgG FcRn L1,2,3a or L1,2,3b were synthesised (GeneArt, ThermoFisher Scientific) with flanking restriction enzyme sites for cloning into Abzena's pANT dual Ig expression vector system for human heavy and kappa light chains. The heavy chains, also containing Trastuzumab VH, were cloned between the Mlu I and Kpnl restriction sites. Trastuzumab Vk, synthesised separately, was cloned between the Pte I and BamH I restriction sites. Individual loop variants were constructed using specific primers to amplify the loop(s) of interest and using pull through PCR to generate IgE with either one or two IgG1 loops in all possible combinations to generate a total of eight additional constructs (containing L1 alone, L2 alone, L3 alone , L1+2, L1+3a, L1+3a, L2+3a and L2+3b).

The 3His variant was generated by site directed mutagenesis using the WT IgE constant domain as template, replacing the relevant residues with Histidine.

To generate IgE-IgG1 CH2 and CH2-CH3 fusion variants, specific primers were used to amplify WT IgE whilst removing the stop codon at the end of IgE CH4 and, in a separate reaction, to amplify either IgG1 CH2 or IgG1 CH2-CH3 which were synthesised separately. Pull through PCR was used to combine both fragments and introduce Mlu I and Kpnl restriction sites for cloning into the dual expression vector.

The following hybrid antibody molecules have been constructed:
IgE containing IgG FcRn Loop 1;
IgE containing IgG FcRn Loop 2;
IgE containing IgG FcRn Loop 3a;
IgE containing IgG FcRn Loop 3b;
IgE containing IgG FcRn Loop 1+Loop 2;
IgE containing IgG FcRn Loop 1+Loop 3a;
IgE containing IgG FcRn Loop 1+Loop 3b;
IgE containing IgG FcRn Loop 2+Loop 3a;
IgE containing IgG FcRn Loop 2+Loop 3b;
IgE containing IgG FcRn Loop 1+Loop 2+Loop 3a;
IgE containing IgG FcRn Loop 1+Loop 2+Loop 3b; and
IgE containing 3×IgG Histidine residue swap only.

In addition, the following fusion proteins have been constructed:
IgE plus IgG1 Hinge-CH2
IgE plus IgG1 Hinge-CH2-CH3

The sequences for wild type Trastuzumab IgE were as follows:

WT IgE_VH_CH1_CH2:
(SEQ ID NO: 1)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCA

WT IgE_CH3 (loops that were replaced are underlined; residues that were replaced with Histidine are in bold italic):

(SEQ ID NO: 2)
DSNPRGVSAYLSRPSPFDLFIRKSPTITCLVVDLAPSKGTVNLTWSRASG
KPVNHSTRKEEKQRNGTLTVTSTLPV [symbol] R
DWIEGETYQCRVTHPHLPRALMRSTTKTS
WT IgE_CH4:
(SEQ ID NO: 3)
GPRAAPEVYAFATPEWPGSRDKRTLACLIQNFMPEDISVQWLHNEVQLPD
ARHSTTQPRKTKGSGFFVFSRLEVTRAEWEQKDEFIC
RAVHEAASPS [symbol] TVQRAVSVNPGK
(SEQ ID NO: 4)
IgE Loop 1: FDLFIRKS
(SEQ ID NO: 5)
IgE Loop 2: PVGTR
(SEQ ID NO: 6)
IgE Loop 3a: ASPSQTV
(SEQ ID NO: 7)
IgE Loop 3b: RAVHEAASPSQTV

Sequences for wild type IgG were as follows:

WT IgG_Hinge:
(SEQ ID NO: 8)
EPKSCDKTHTCPPCP

WT IgG_CH2 (loops italicised and underlined; substituted Histidine in bold):

(SEQ ID NO: 9)
APELLGGPSVFLFPPKPKDTLMISRTPEVTCVVVDVSHEDPEVKFNWYVD
GVEVHNAKTKPREEQYNSTYRVVSVTVL [symbol] DWLNGKEYKCKVSNK
ALPAPIEKTISKAK
WT IgG_CH3:
(SEQ ID NO: 10)
GQPREPQVYTLPPSREEMTKNQVSLTCLVKGFYPSDIAVEWESNGQPENN
YKTTPPVLDSDGSFFLYSKLTVDKSRWQQGNVFSCSVMHEA [symbol]
QKSLSLSPGK
(SEQ ID NO: 11)
IgG FcRn-binding Loop 1: KDTLMISRT
(SEQ ID NO: 12)
IgG FcRn-binding Loop 2: TVLHQ
(SEQ ID NO: 13)
IgG FcRn-binding Loop 3a: LHNHYT
(SEQ ID NO: 14)
IgG FcRn-binding Loop 3: SVMHEALHNHYT

Sequences for the hybrid molecules were as follows. Each hybrid molecule further comprises wild-type IgE_VH_CH1CH2 (i.e. SEQ ID NO:1):

IgE_CH3_CH4 containing IgG FcRn-binding Loop 1:
(SEQ ID NO: 15)
DSNPRGVSAYLSRPSPKDTLMISRTPTITCLVVDLAPSKGTVNLTWSRAS
GKPVNHSTRKEEKQRNGTLTVTSTLPVGTRDWIEGETYQCRVTHPHLPRA
LMRSTTKTSGPRAAPEVYAFATPEWPGSRDKRTLACLIQNFMPEDISVQW
LHNEVQLPDARHSTTQPRKTKGSGFFVFSRLEVTRAEWEQKDEFICRAVH
EAASPSQTVQRAVSVNPGK
IgE_CH3_CH4 containing IgG FcRn-binding Loop 2:
(SEQ ID NO: 16)
DSNPRGVSAYLSRPSPFDLFIRKSPTITCLVVDLAPSKGTVNLTWSRASG
KPVNHSTRKEEKQRNGTLTVTSTLTVLHQDWIEGETYQCRVTHPHLPRAL
MRSTTKTSGPRAAPEVYAFATPEWPGSRDKRTLACLIQNFMPEDISVQWL
HNEVQLPDARHSTTQPRKTKGSGFFVFSRLEVTRAEWEQKDEFICRAVHE
AASPSQTVQRAVSVNPGK
IgE_CH3_CH4 containing IgG FcRn-binding Loop 3a:
(SEQ ID NO: 17)
DSNPRGVSAYLSRPSPFDLFIRKSPTITCLVVDLAPSKGTVNLTWSRASG
KPVNHSTRKEEKQRNGTLTVTSTLPVGTRDWIEGETYQCRVTHPHLPRAL
MRSTTKTSGPRAAPEVYAFATPEWPGSRDKRTLACLIQNFMPEDISVQWL
HNEVQLPDARHSTTQPRKTKGSGFFVFSRLEVTRAEWEQKDEFICRAVHE
ALHNHYTQRAVSVNPGK
IgE_CH3_CH4 containing IgG FcRn-binding Loop 3b:
(SEQ ID NO: 18)
DSNPRGVSAYLSRPSPFDLFIRKSPTITCLVVDLAPSKGTVNLTWSRASG
KPVNHSTRKEEKQRNGTLTVTSTLPVGTRDWIEGETYQCRVTHPHLPRAL
MRSTTKTSGPRAAPEVYAFATPEWPGSRDKRTLACLIQNFMPEDISVQWL
HNEVQLPDARHSTTQPRKTKGSGFFVFSRLEVTRAEWEQKDEFICSVMHE
ALHNHYTQRAVSVNPGK
IgE_CH3_CH4 containing IgG FcRn-binding Loop
1 + Loop 2:
(SEQ ID NO: 19)
DSNPRGVSAYLSRPSPKDTLMISRTPTITCLVVDLAPSKGTVNLTWSRAS
GKPVNHSTRKEEKQRNGTLTVTSTLTVLHQDWIEGETYQCRVTHPHLPRA
LMRSTTKTSGPRAAPEVYAFATPEWPGSRDKRTLACLIQNFMPEDISVQW
LHNEVQLPDARHSTTQPRKTKGSGFFVFSRLEVTRAEWEQKDEFICRAVH
EAASPSQTVQRAVSVNPGK
IgE_CH3_CH4 containing IgG FcRn-binding Loop
1 + Loop 3a:
(SEQ ID NO: 20)
DSNPRGVSAYLSRPSPKDTLMISRTPTITCLVVDLAPSKGTVNLTWSRAS
GKPVNHSTRKEEKQRNGTLTVTSTLPVGTRDWIEGETYQCRVTHPHLPRA
LMRSTTKTSGPRAAPEVYAFATPEWPGSRDKRTLACLIQNFMPEDISVQW
LHNEVQLPDARHSTTQPRKTKGSGFFVFSRLEVTRAEWEQKDEFICRAVH
EALHNHYTQRAVSVNPGK
IgE_CH3_CH4 containing IgG FcRn-binding Loop
1 + Loop 3b:
(SEQ ID NO: 21)
DSNPRGVSAYLSRPSPKDTLMISRTPTITCLVVDLAPSKGTVNLTWSRAS
GKPVNHSTRKEEKQRNGTLTVTSTLPVGTRDWIEGETYQCRVTHPHLPRA
LMRSTTKTSGPRAAPEVYAFATPEWPGSRDKRTLACLIQNFMPEDISVQW
LHNEVQLPDARHSTTQPRKTKGSGFFVFSRLEVTRAEWEQKDEFICSVMH
EALHNHYTQRAVSVNPGK
IgE_CH3_CH4 containing IgG FcRn-binding Loop
2 + Loop 3a:
(SEQ ID NO: 22)
DSNPRGVSAYLSRPSPFDLFIRKSPTITCLVVDLAPSKGTVNLTWSRASG
KPVNHSTRKEEKQRNGTLTVTSTLTVLHQDWIEGETYQCRVTHPHLPRAL
MRSTTKTSGPRAAPEVYAFATPEWPGSRDKRTLACLIQNFMPEDISVQWL
HNEVQLPDARHSTTQPRKTKGSGFFVFSRLEVTRAEWEQKDEFICRAVHE
ALHNHYTQRAVSVNPGK
IgE_CH3_CH4 containing IgG FcRn-binding Loop
2 + Loop 3b:
(SEQ ID NO: 23)
DSNPRGVSAYLSRPSPFDLFIRKSPTITCLVVDLAPSKGTVNLTWSRASG
KPVNHSTRKEEKQRNGTLTVTSTLTVLHQDWIEGETYQCRVTHPHLPRAL
MRSTTKTSGPRAAPEVYAFATPEWPGSRDKRTLACLIQNFMPEDISVQWL
HNEVQLPDARHSTTQPRKTKGSGFFVFSRLEVTRAEWEQKDEFICSVMHE
ALHNHYTQRAVSVNPGK
IgE_CH3_CH4 containing IgG FcRn Loop 1 +
Loop 2 + Loop 3a:
(SEQ ID NO: 24)
DSNPRGVSAYLSRPSPKDTLMISRTPTITCLVVDLAPSKGTVNLTWSRAS
GKPVNHSTRKEEKQRNGTLTVTSTLTVLHQDWIEGETYQCRVTHPHLPRA
LMRSTTKTSGPRAAPEVYAFATPEWPGSRDKRTLACLIQNFMPEDISVQW
LHNEVQLPDARHSTTQPRKTKGSGFFVFSRLEVTRAEWEQKDEFICRAVH
EALHNHYTQRAVSVNPGK
IgE_CH3_CH4 containing IgG FcRn Loop 1 +
Loop 2 + Loop 3b:
(SEQ ID NO: 25)
DSNPRGVSAYLSRPSPKDTLMISRTPTITCLVVDLAPSKGTVNLTWSRAS
GKPVNHSTRKEEKQRNGTLTVTSTLTVLHQDWIEGETYQCRVTHPHLPRA
LMRSTTKTSGPRAAPEVYAFATPEWPGSRDKRTLACLIQNFMPEDISVQW
LHNEVQLPDARHSTTQPRKTKGSGFFVFSRLEVTRAEWEQKDEFICSVMH
EALHNHYTQRAVSVNPGK
IgE_CH3_CH4 3His
(SEQ ID NO: 26)
DSNPRGVSAYLSRPSPFDLFIRKSPTITCLVVDLAPSKGTVNLTWSRASG
KPVNHSTRKEEKQRNGTLTVTSTLPVGHRDWIEGETYQCRVTHPHLPRAL
MRSTTKTSGPRAAPEVYAFATPEWPGSRDKRTLACLIQNFMPEDISVQWL
HNEVQLPDARHSTTQPRKTKGSGFFVFSRLEVTRAEWEQKDEFICRAVHE
AAHPSHTVQRAVSVNPGK

Sequences for the fusion proteins were as follows. Each fusion protein further comprises wild-type IgE_VH_CH1_CH2 and IgE_CH3 (i.e. SEQ ID NOs:1 and 2):

IgE_CH4 plus IgG1 Hinge_CH2 (containing RS
linker):
(SEQ ID NO: 27)
GPRAAPEVYAFATPEWPGSRDKRTLACLIQNFMPEDISVQWLHNEVQLPD
ARHSTTQPRKTKGSGFFVFSRLEVTRAEWEQKDEFICRAVHEAASPSQTV
QRAVSVNPGKRSEPKSCDKTHTCPPCPAPELLGGPSVFLFPPKPKDTLMI
SRTPEVTCVVVDVSHEDPEVKFNWYVDGVEVHNAKTKPREEQYNSTYRVV
SVLTVL [symbol] DWLNGKEYKCKVSNKALPAPIEKTISKAK
IgE_CH4 plus IgG1 Hinge_CH2_CH3 (containing RS
linker)
(SEQ ID NO: 28)
GPRAAPEVYAFATPEWPGSRDKRTLACLIQNFMPEDISVQWLHNEVQLPD
ARHSTTQPRKTKGSGFFVFSRLEVTRAEWEQKDEFICRAVHEAASPSQTV
QRAVSVNPGKRSEPKSCDKTHTCPPCPAPELLGGPSVFLFPPKPKDTLMI
SRTPEVTCVVVDVSHEDPEVKFNYVDGVEVHNAKTKPREEQYNSTYRVVW
SVLTVL [symbol] DWLNGKEYKCKVSNKALPAPIEKTISKAKGQPREPQVYTLPP
SREEMTKNQVSLTCLVKGFYPSDIAVEWESNGQPENNYKTTPPVLDSDGS
FFLYSKLTVDKSRWOOGNVFSCSVMHE [symbol] QKSLSLSPGK

The full amino acid sequence of the heavy chain of the IgE plus IgG1 Hinge_CH2 construct is shown below:

(SEQ ID NO: 29)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGKRS
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAK

The full amino acid sequence of the heavy chain of the IgE plus IgG1 Hinge_CH2_CH3 construct is shown below:

(SEQ ID NO: 30)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGKRS
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK

The following mutant loop sequences are found in CH3 and CH4 domains of the IgE 3His construct:

(SEQ ID NO: 31)
IgE Loop 2: PVGHR
(SEQ ID NO: 32)
IgE Loop 3a: AHPSHTV
(SEQ ID NO: 33)
IgE Loop 3b: RAVHEAAHPSHTV

The full amino acid sequence of the heavy chain of the IgE 3His construct is shown below (i.e. WT IgE_VH_CH1_CH2 plus IgE_CH3_CH4 3His):

(SEQ ID NO: 34)
EVQLVESGGGLVQPGGSLRLSCAASGFNKKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGHRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAAHPSHTVQRAVSVNPGK

The full amino acid sequence of the light chain of the IgE 3His construct (and other constructs disclosed herein) is shown below:

(SEQ ID NO: 35)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKGTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

All constructs were confirmed by sequencing. DNA was prepared and transiently transfected into CHO cells using the MaxCyte STX® electroporation system (MaxCyte Inc., Gaithersburg, USA) with OC-400 processing assemblies. 7-10 days post transfection, the supernatants were harvested.

Antibodies (i.e. comprising the variant heavy chains described above and kappa light chains derived from trastuzumab IgE) were purified from cell culture supernatant using either CaptureSelect™ IgE Affinity Matrix (ThermoFisher, Loughborough, UK) or Mab Select Sure columns (GE Healthcare, Little Chalfont, UK) for the IgG1 CH2-CH3 fusion. Eluted fractions were buffer exchanged into PBS and filter sterilised before quantification by A28onm using an extinction coefficient (E_{c (0.1%)}) based on the predicted amino acid sequence.

### Example 2

### Binding of IgE variants to FcRn

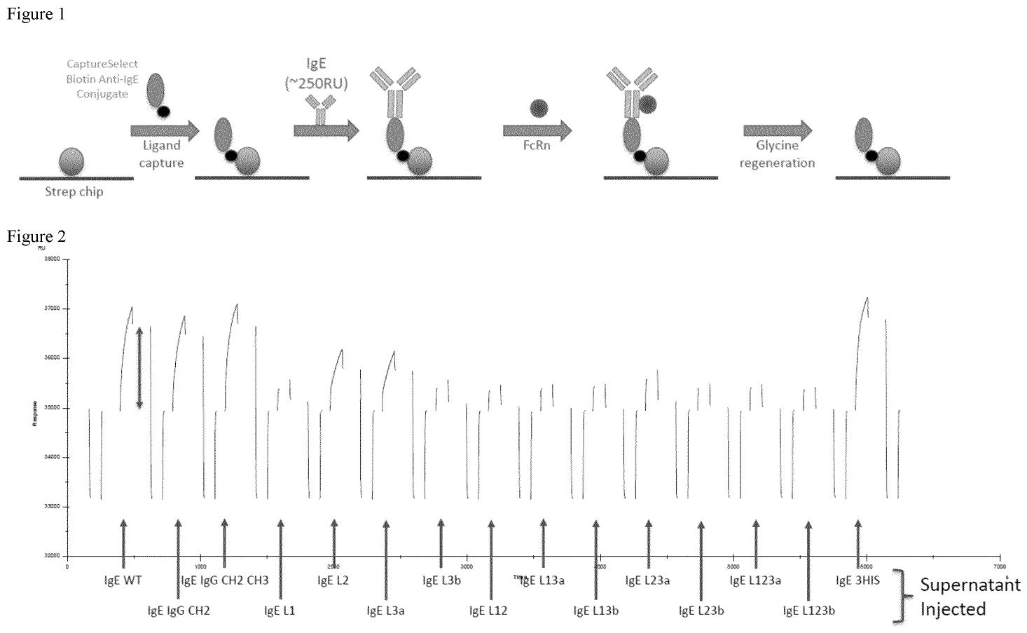

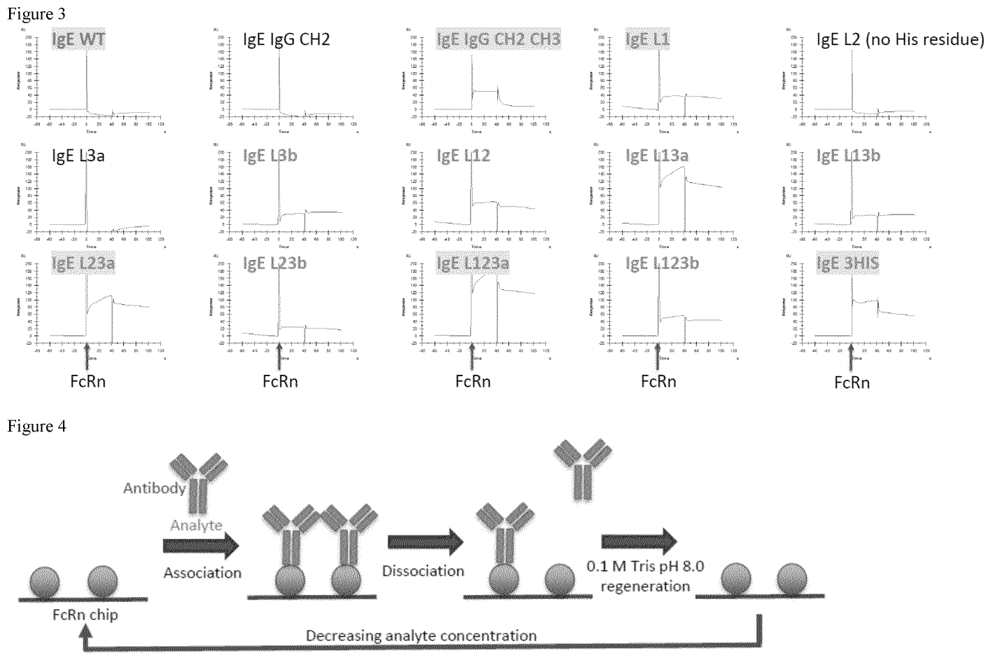

To assess the binding of the antibody variants to FcRn (Sino Biological Cat. No. CT009-H08H), Biacore kinetic analysis at a single concentration was performed on supernatants from transfected CHO cell cultures. Kinetic experiments were performed on a Biacore T200 (serial no. 1909913) running Biacore T200 Control software V2.0.1 and Evaluation software V3.0 (GE Healthcare, Uppsala, Sweden). The principle of the assay is shown in FIG. 1. All kinetic experiments were run at 25° C. with PBS containing 0.05% P20 (GE Healthcare, Little Chalfont, UK) and an additional 150 mM NaCl (pH 6.0). Antibodies were loaded onto F_{c}2, F_{c}3 and F_{c}4 of the Straptavidin chip (GE Healthcare, Little Chalfont, UK) preloaded with CaptureSelect Biotin Anti-IgE (Thermo Cat. No. 7103542500). Antibodies were captured at a flow rate of 10 μl/min to give an immobilisation level (RL) of ˜250RU. Binding data was obtained with FcRn at 2000 nM for 40 seconds at a flow rate of 10 μl/min. Wild-type IgE was used as a negative control. The signal from the reference channel F_{c}1 (no antibody) was subtracted from that of F_{c}2, F_{c}3 and F_{c}4 to correct for differences in non-specific binding to a reference surface. Regeneration of the anti-IgE capture surface was conducted using one injection of glycine pH 2.0.

As can be seen in FIG. 2, a significant difference in the level of antibody captured was seen. The amount captured with variants containing either Loop 1 or 3b or two loop swaps appeared much lower than that observed for wild-type IgE the IgG fusion antobodies or the 3His substitution antibodies. Reasons for this may be that expression is low or capture is less efficient. Dilution and contact times were adjusted to allow sufficient loading during the FcRn binding run.

As can be seen in FIG. 3, differences in binding were observed for the variants, although a number of variants are of interest to pursue further. In general, the control proteins appear to behave as expcted, with no binding of wild-type IgE being seen while binding of IgE-IgG_CH2_CH3 was observed. There may be some binding to the reference F_{c}1, leading to a drift below baseline for some of the IgE variants.

Using non-purified proteins, the binding kinetics appear different to that observed for the fusion protein IgE_IgG_CH2_CH3. The binding profile of the fusion protein IgE_IgG_CH2_CH3 is similar to results that would be expected from an assay that was run with FcRn coupled to the chip, instead of the other way around. With purified antibodies, it is typical to immobilise FcRn on the chip using standard amine chemistry and to flow over different concentrations of antibody. As the concentration of the IgEs in the supernatant was unknown, this approach is not suitable.

If binding to CaptureSelect was low, an alternative purification may be needed. If expression was low, it was surmised that large volumes of cells may be required to generate sufficient antibody for purification and further analysis. However, purification using an anti-kappa select resin, together with preparaticve size exclusion chromatography (SEC) suggest that expression is not an issue (not shown).

Based on these results, a decision was made to purify and re-test the majority of the variants using purified material in a standard assay set up.

### Example 3

### Binding of Purified Hybrid IgE Variants

The aim of this experiment was to assess the binding of purified IgE variant antibodies to human FcRn. Wild-type IgE was used as a negative control and Herceptin was used as a positive control.

The binding of IgG to FcRn is pH dependent and is involved in recycling of antibodies taken up into the endosome back into the serum. FcRn has a higher affinity for IgG at pH 6.0 than at pH 7.4.

To determine the kinetics of selected variants to FcRn, multi cycle kinetic analysis was performed on purified antibodies. Kinetic experiments were performed on a Biacore T200 (serial no. 1909913) running Biacore T200 Control software V2.0.1 and Evaluation software V3.0 (GE Healthcare, Uppsala, Sweden). All kinetic experiments were run at 25° C. with PBS containing 0.05% P20 (GE Healthcare, Little Chalfont, UK) and an additional 150 mM NaCl (pH 6.0 or pH 7.4). The principle of the assay is shown in FIG. 1. Human FcRn was directly coupled to a CM5 chip (GE Healthcare, Little Chalfont, UK) using standard amine chemistry to ˜300 RU. Multi cycle kinetic data was obtained with purified antibody as the analyte at a flow rate of 30 μl/min to minimise any potential mass transport limitations. A five point, three-fold dilution range from 24.7 nM to 2000 nM of antibody was used for pH 6.0 analysis, for pH 7.4 analysis a three point, three-fold dilution range from 222.2 nM to 2000 nM of antibody was used. The association phase for the injections of antibody was monitored for 25 seconds and the dissociation phase was measured for 75 seconds. Regeneration of the FcRn surface was performed using 0.1M Tris pH 8.0 injections. The signal from the reference channel F_{c}1 was subtracted to correct for differences in non-specific binding to a reference surface, and a steady state binding model used to fit the data.

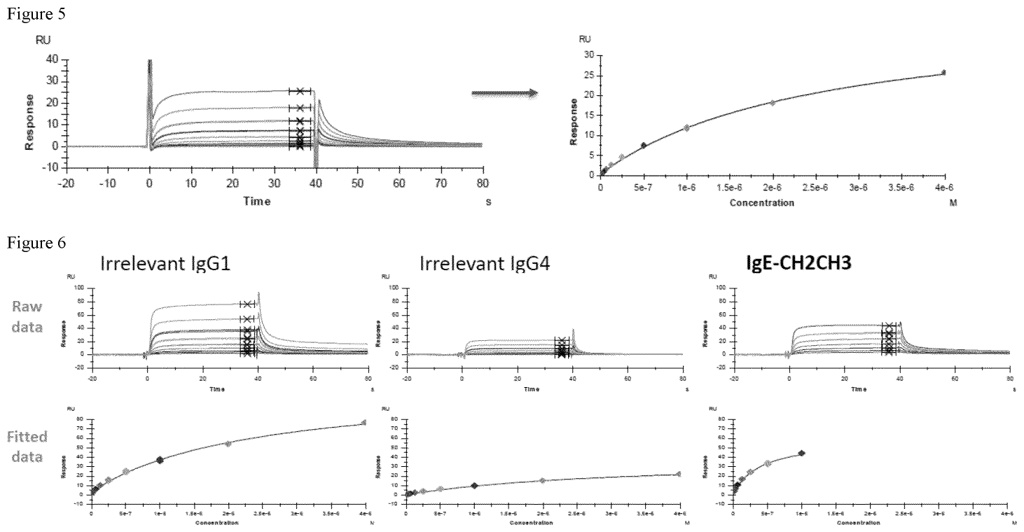

Steady state analysis was carried out on the resulting data, such analysis being particularly suitable for low affinity interactions. A plot of the response at equilibrium (R_{eq}) is plotted against concentration. For affinity measurements, a sensorgram should reach a steady state (plateau at X) during the association phase of binding (see FIG. 5). On a plot of response vs concentration, the K_{D} value is equal to the concentration that gives 50% of the maximum response. K_{D} is provided where a reasonable curvature was obtained when response at equilibrium (R_{eq}) was plotted against concentration.

FIG. 6 and Table 1 show binding of IgG1, IgG4 and the fusion construct IgE_IgG_CH2_CH3 to FcRn at pH 6.0.

TABLE 1

| | | | | Relative |
|---|---|---|---|---|
| Antibody | K_{D}(M) | R_{MAX}(RU) | Chi²(RU²) | Binding |
| Irrelevant IgG1 | 2.37E−06 | 114.5 | 2.38 | ++ |
| Irrelevant IgG4 | 3.25E−06 | 38.8 | 0.0748 | ++ |
| IgE_CH2_CH3* | 4.22E−07 | 57.7 | 0827 | +++ |

*Top two concetrations were removed due to the shape of the sensorgram

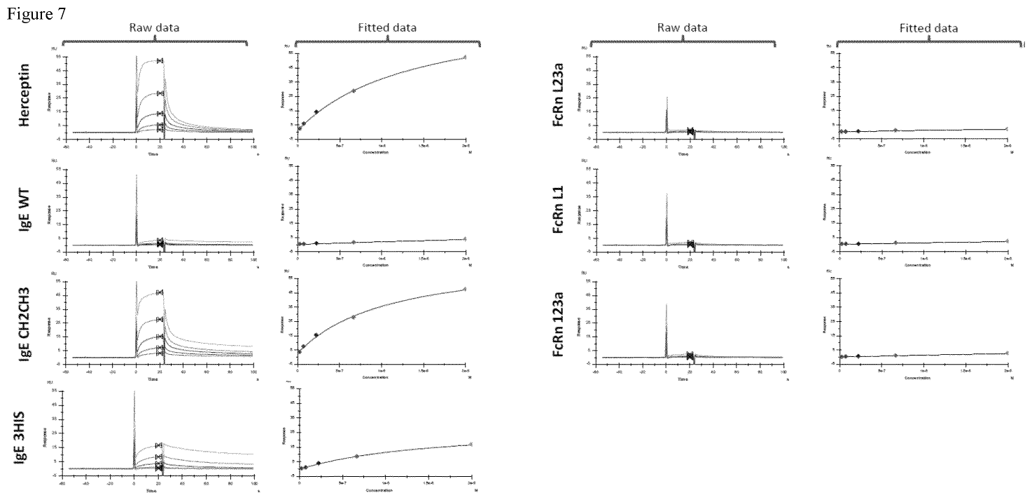

FIG. 7 and Table 2 show the raw and fitted data for binding of Herceptin, wild-type IgE, IgE_IgG_CH2_CH3, IgE containing 3× IgG Histadine residues, IgE containing IgG FcRn Loop 2 and Loop 3a, IgE containing IgG FcRn Loop 1 and IgE containing IgG FcRn Loop 1, Loop 2 and Loop 3a to human FcRn at pH 6.0.

TABLE 2

| Antibody | K_{D}(M) | R_{MAX}(RU) | Chi²(RU²) |
|---|---|---|---|
| Herceptin | 1.39E−06 | 86.2 | 0.620 |
| wild-type IgE | N/A | | |
| IgE_IgG_CH2_CH3 | 1.10E−06 | 68.9 | 1.500 |
| IgE 3His | 1.62E−06 | 30.2 | 0.0053 |
| FcRn L23a | N/A | | |
| FcRn L1 | N/A | | |
| FcRn L123a | N/A | | |

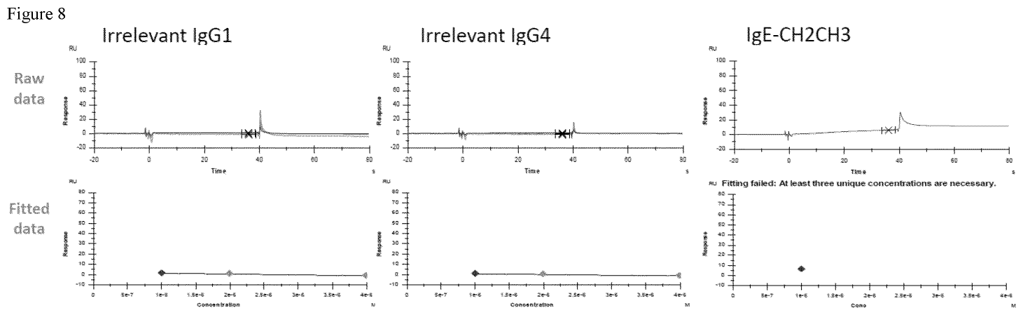

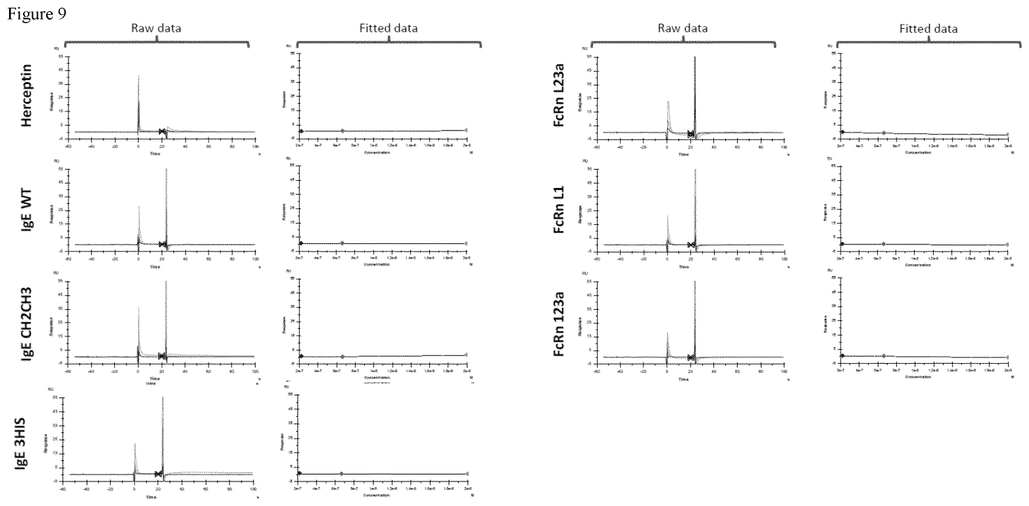

FIGS. 8 and 9 and Tables 3 and 4 show the results of the same experiment carried out at pH7.4.

TABLE 3

| | | | | Relative |
|---|---|---|---|---|
| Antibody | K_{D}(M) | R_{MAX}(RU) | Chi²(RU²) | Binding |
| Irrelevant IgG1 | — | — | — | — |
| Irrelevant IgG4 | — | — | — | — |
| IfE_CH2_CH3* | — | — | — | — |

*Top two concetrations were removed due to the shape of the sensorgram

TABLE 4

| Antibody | K_{D}(M) | R_{MAX}(RU) | Chi²(RU²) |
|---|---|---|---|
| Herceptin | — | — | — |
| wild-type IgE | — | — | — |
| IgE_IgG_CH2_CH3 | — | — | — |
| IgE 3His | — | — | — |
| FcRn L23a | — | — | — |
| FcRn L1 | — | — | — |
| FcRn L123a | — | — | — |

As can be seen, the binding of IgE_IgG_CH2_CH3 to FcRn is broadly similar to that of wild-type IgG.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

### Example 4

### Anti-HMW-MAA Hybrid Antibody

In a further example, another IgE 3His variant is created (see Example 1, SEQ ID NOs: 34 and 35). In this example, the IgE antibody is based on an anti-HMW-MAA antibody, for example, as disclosed in WO 2013/050725, rather than trastuzumab IgE as in Example 1. Thus in this example, the trastuzumab VH and VL domains (as present in SEQ ID NOs: 34 and 35) are replaced with anti-HMW-MAA VH and VL domains.The antibodies are produced and purified as described in Example 1. Analysis of antibody binding is tested as described in Examples 2-3.

The variable domain sequences for a HMW-MAA IgE are as follows:

HMW-MAA VH (SEQ ID NO: 170):
EQVKLQQSGGGLVQPGGSMKLSCVVSGFTFSNYWMNWVRQSPEKGLEWIA
EIRLKSNNFGRYYAESVKGRFTISRDDSKSSAYLQMINLRAEDTGIYYCT
SYGNYVGHYFDHWGQGTTVTVSS
HMW-MAA VL (SEQ ID NO: 171):
DIELTQSPKFMSTSVCDRVSVTCKASQNVDTNVAWYQQKPGQSPEPLLFS
ASYRYTGVPDRFTGSGSGTDFTLTISNVQSEDLAEYFCQQYNSYPLTFGG
GTKLEIK

In an alternative embodiment, the variable domain sequences for a HMW-MAA IgE are as follows:

HMW-MAA VH (SEQ ID NO: 184):
EVQLVQSGGGLVQPGGSLKLSCAVSGFTFSNYWMNWVRQAPGKGLEWVGE
IRLKSNNFGRYYAESVKGRFTISRDDSKNTAYLQMNSLKTEDTAVYYCTS
YGNYVGHYFDHWGQGTLVTVSS
HMW-MAA VL (SEQ ID NO: 185):
DIQLTQSPSFLSASVGDRVTITCKASQNVDTNVAWYQQKPGKAPKPLLFS
ASYRYTGVPSRFSGSGSGTDFTLTISSLQPEDFATYFCQQYNSYPLTFGG
GTKVEIK

Thus in specific embodiments, the anti-HMW-MAA may comprise one of the following heavy or light chain sequences (underlining shows variable domain sequences, standard text shows IgE Fc sequences, bold underline sequences indicate a His mutation):

HMW-MAA heavy chain (SEQ ID NO: 186):
EQVKLQQSGGGLVQPGGSMKLSCVVSGFTFSNYWMNWVRQSPEKGLEWIA
EIRLKSNNFGRYYAESVKGRFTISRDDSKSSAYLQMINLRAEDTGIYYCT
SYGNYVGHYFDHGQGTTVTVSSASTQSPSVFPLTRCCKNIPSNATSWVTL
GCLATGYFPEPVMVTWDTGSLNGTTMTLPATTLTLSGHYATISLLTVSGA
WAKQMFTCRVAHTPSSTDWVDNKTFSVCSRDFTPPTVKILQSSCDGGGHF
PPTIQLLCLVSGYTPGTINITWLEDGQVMDVDLSTASTTQEGELASTQSE
LTLSQKHWLSDRTYTCQVTYQGHTFEDSTKKCADSNPRGVSAYLSRPSPF
DLFIRKSPTITCLVVDLAPSKGTVNLTWSRASGKPVNHSTRKEEKQRNGT
LTVTSTLPVGHRDWIEGETYQCRVTHPHLPRALMRSTTKTSGPRAAPEVY
AFATPEWPGSRDKRTLACLIQNFMPEDISVQWLHNEVQLPDARHSTTQPR
KTKGSGFFVFSRLEVTRAEWEQKDEFICRAVHEAAHPSHTVQRAVSVNPG
K
HMW-MAA light chain (SEQ ID NO: 187):
DIELTQSPKFMSTSVCDRVSVTCKASQNVDTNVAWYQQKPGQSPEPLLFS
ASYRYTGVPDRFTGSGSGTDFTLTISNVQSEDLAEYFCQQYNSYPLTFGG
GTKLEIKGTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Alternative HMW-MAA heavy chain (SEQ ID NO: 188):
EVQLVQSGGGLVQPGGSLKLSCAVSGFTFSNYWMNWVRQAPGKGLEWVGE
IRLKSNNFGRYYAESVKGRFTISRDDSKNTAYLQMNSLKTEDTAVYYCTS
YGNYVGHYFDHWGQGTLVTVSSASTQSPSVFPLTRCCKNIPSNATSVTLG
CLATGYFPEPVMVTWDTGSLNGTTMTLPATTLTLSGHYATISLLTVSGAW
AKQMFTCRVAHTPSSTDWVDNKTFSVCSRDFTPPTVKILQSSCDGGGHFP
PTIQLLCLVSGYTPGTINITWLEDGQVMDVDLSTASTTQEGELASTQSEL
TLSQKHWLSDRTYTCQVTYQGHTFEDSTKKCADSNPRGVSAYLSRPSPFD
LFIRKSPTITCLVVDLAPSKGTVNLTWSRASGKPVNHSTRKEEKQRNGTL
TVTSTLPVGHRDWIEGETYQCRVTHPHLPRALMRSTTKTSGPRAAPEVYA
FATPEWPGSRDKRTLACLIQNFMPEDISVQWLHNEVQLPDARHSTTQPRK
TKGSGFFVFSRLEVTRAEWEQKDEFICRAVHEAAHPSHTVQRAVSVNPGK
Alternative HMW-MAA light chain (SEQ ID NO: 189):
DIQLTQSPSFLSASVGDRVTITCKASQNVDTNVAWYQQKPGKAPKPLLFS
ASYRYTGVPSRFSGSGSGTDFTLTISSLQPEDFATYFCQQYNSYPLTFGG
GTKVEIKGTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### Example 5

### Production of a Heterodimeric IgE

Construction of IgE-IgG-Fc (IGEG) fusion proteins

DNA sequences corresponding to the WT IgE constant domain were codon optimised for CHO expression and synthesised (GeneArt, ThermoFisher Scientific, Loughborough, UK) with flanking restriction enzyme sites for cloning into a pANT dual Ig expression vector system for human heavy and kappa light chains. The heavy chain, also containing Trastuzumab VH, was cloned between the Mlu I and Kpn I restriction sites. Trastuzumab Vk, synthesised separately, was cloned between the BssH II and BamH I restriction sites, upstream of the kappa constant region.

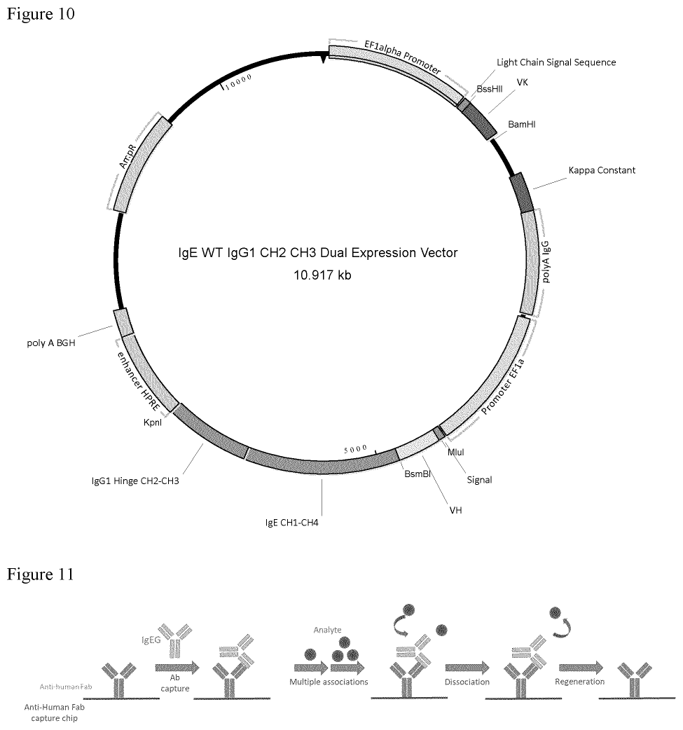

In order to generate the IgE-IgG (IGEG) fusion, specific primers were used to amplify WT IgE whilst removing the stop codon at the end of IgE CH4, and in a separate reaction to amplify IgG1 Hinge-CH2-CH3 synthesised separately. Pull-through PCR was used to combine both fragments and introduce Mlu I and KpnI restriction sites for cloning into the dual expression vector. A BsmBI restriction site was subsequently introduced by site directed mutagenesis (Quikchange, Agilent) within the FW4 region of the Trastuzumab VH which, along with Mlu I, permitted swapping of VH regions (See FIG. 10 for a diagram of the vector).

To remove a potential free cysteine residue within the IgG hinge region, primers were designed to introduce the Cys220Ser amino acid substitutions (numbering is based upon the EU numbering scheme with reference to the IgG portion of the IGEG sequence) by site directed mutagenesis using the BsmBI-containing IgE-IgG construct as template. The Cys220Ser mutation is indicated in blue in the sequences below.

To remove the ability of the IgG portion of the IGEG to bind to FcRn, amino acid substitutions were made at three residues normally involved in FcRn binding, Ile253Ala, His310Ala and His435Ala (numbering is based upon the EU numbering scheme with reference to the IgG portion of the IGEG sequence). Primers were designed and site directed mutagenesis (Agilent Quikchange) performed using the BsmBI-containing IgE-IgG constructs (containing either Cys220 or Ser220) as template.

In order to generate the CH1 series of constructs, the CH1 VH and VK were synthesised (GeneArt) and cloned into the IGEG vectors. The CH1 VH was cloned between the MluI and BsmBI restriction sites, and the CH1 Vk was cloned between the BssH II and BamH I restriction sites.

All constructs were confirmed by Sanger sequencing.

The sequences were as follows (underlining shows variable domain sequences, standard text shows IgE Fc sequences, bold shows IgG-derived sequences, bold underline shows specific mutations):

Trastuzumab IgE/IGEG Variant Sequences
Trastuzumab IgE Heavy Chain
(SEQ ID NO: 172)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGK
Trastuzumab IgE-IgG-Fc Heavy Chain
(SEQ ID NO: 173)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEPGSRDKRTLACLIQNFMPEDISVQWLHNEVQLPDARHSTTQPRKTKW
GSGFFVFSRLEVTRAEWEQKDEFICRAVHEAASPSQTVQRAVSVNPGKRS
EPKSCDKTHTCPPCPAPELLGGPSVFLFPPKPKDT
LMISRTPEVTCVVVDVSHEDPEVKFNWYVDGVEVH
NAKTKPREEQYNSTYRVVSVLTVLHQDWLNGKEYK
CKVSNKALPAPIEKTISKAKGQPREPQVYTLPPSR
EEMTKNQVSLTCLVKGFYPSDIAVEWESNGQPENN
YKTTPPVLDSDGSFFLYSKLTVDKSRWQQGNVFSC
SVMHEALHNHYTQKSLSLSPGK
Trastuzumab IgE-IgG-Fc C220S Heavy Chain
(SEQ ID NO: 174)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGKRS
EPKSSDKTHTCPPCPAPELLGGPSVFLFPPKPKDT
LMISRTPEVTCVVVDVSHEDPEVKFNWYVDGVEVH
NAKTKPREEQYNSTYRVVSVLTVLHQDWLNGKEYK
CKVSNKALPAPIEKTISKAKGQPREPQVYTLPPSR
EEMTKNQVSLTCLVKGFYPSDIAVEWESNGQPENN
YKTTPPVLDSDGSFFLYSKLTVDKSRWQQGNVFSC
SVMHEALHNHYTQKSLSLSPGK
Trastuzumab IgG-IgG-Fc dFcRn Heavy Chain
(SEQ ID NO: 175)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGKRS
EPKSCDKTHTCPPCPAPELLGGPSVFLFPPKPKDT
LMASRTPEVTCVVVDVSHEDPEVKFNWYVDGVEVH
NAKTKPREEQYNSTYRVVSVLTVLAQDWLNGKEYK
CKVSNKALPAPIEKTISKAKGQPREPQVYTLPPSR
EEMTKNQVSLTCLVKGFYPSDIAVEWESNGQPENN
YKTTPPVLDSDGSFFLYSKLTVDKSRWQQGNVFSC
SVMHEALHNAYTQKSLSLSPGK
Trastuzumab IgG-IgG-Fc dFcRn C220S Heavy Chain
(SEQ ID NO: 176)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTQSPSVFPLTRCCKNIPSNATSVTLGCL
ATGYFPEPVMVTWDTGSLNGTTMTLPATTLTLSGHYATISLLTVSGAWAK
QMFTCRVAHTPSSTDWVDNKTFSVCSRDFTPPTVKILQSSCDGGGHFPPT
IQLLCLVSGYTPGTINITWLEDGQVMDVDLSTASTTQEGELASTQSELTL
SQKHWLSDRTYTCQVTYQGHTFEDSTKKCADSNPRGVSAYLSRPSPFDLF
IRKSPTITCLVVDLAPSKGTVNLTWSRASGKPVNHSTRKEEKQRNGTLTV
TSTLPVGTRDWIEGETYQCRVTHPHLPRALMRSTTKTSGPRAAPEVYAFA
TPEWPGSRDKRTLACLIQNFMPEDISVQWLHNEVQLPDARHSTTQPRKTK
GSGFFVFSRLEVTRAEWEQKDEFICRAVHEAASPSQTVQRAVSVNPGKRS
EPKSSDKTHTCPPCPAPELLGGPSVFLFPPKPKDT
LMASRTPEVTCVVVDVSHEDPEVKFNWYVDGVEVH
NAKTKPREEQYNSTYRVVSVLTVLAQDWLNGKEYK
CKVSNKALPAPIEKTISKAKGQPREPQVYTLPPSR
EEMTKNQVSLTCLVKGFYPSDIAVEWESNGQPENN
YKTTPPVLDSDGSFFLYSKLTVDKSRWQQGNVFSC
SVMHEALHNAYTQKSLSLSPGK
Kappa Trastuzumab Light Chain
(SEQ ID NO: 177)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECVKCK
HMW-MAA IgE/IGEG Variant Sequences
HMW-MAA IgE Heavy Chain
(SEQ ID NO: 178)
EVQLVQSGGGLVQPGGSLKLSCAVSGFTFSNYWMNWVRQAPGKGLEWVGE
IRLKSNNFRYYAESVKGRFTISRDDSKNTAYLQMNSLKTEDTAVYYCTSY
GNYVGHYFDHWGQGTLVTVSSASTQSPSVFPLTRCCKNIPSNATSVTLGC
LATGYFPEPVMVTWDTGSLNGTTMTLPATTLTLSGHYATISLLTVSGAWA
KQMFTCRVAHTPSSTDWVDNKTFSVCSRDFTPPTVKILQSSCDGGGHFPP
TIQLLCLVSGYTPGTINITWLEDGQVMDVDLSTASTTQEGELASTQSELT
LSQKHWLSDRTYTCQVTYQGHTFEDSTKKCADSNPRGVSAYLSRPSPFDL
FIRKSPTITCLVVDLAPSKGTVNLTWSRASGKPVNHSTRKEEKQRNGTLT
VTSTLPVGTRDWIEGETYQCRVTHPHLPRALMRSTTKTSGPRAAPEVYAF
ATPEWPGSRDKRTLACLIQNFMPEDISVQWLHNEVQLPDARHSTTQPRKT
KGSGFFVFSRLEVTRAEWEQKDEFICRAVHEAASPSQTVQRAVSVNPGK
HMW-MAA IgE IgG-Fc Heavy Chain
(SEQ ID NO: 179)
EVQLVQSGGGLVQPGGSLKLSCAVSGFTFSNYWMNWVRQAPGKGLEWVGE
IRLKSNNFGRYYAESVKGRFTISRDDSKNTAYLQMNSLKTEDTAVYYCTS
YGNYVGHYFDHWGQGTLVTVSSASTQSPSVFPLTRCCKNIPSNATSVTLG
CLATGYFPEPVMVTWDTGSLNGTTMTLPATTLTLSGHYATISLLTVSGAW
AKQMFTCRVAHTPSSTDWVDNKTFSVCSRDFTPPTVKILQSSCDGGGHFP
PTIQLLCLVSGYTPGTINITWLEDGQVMDVDLSTASTTQEGELASTQSEL
TLSQKHWLSDRTYTCQVTYQGHTFEDSTKKCADSNPRGVSAYLSRPSPFD
LFIRKSPTITCLVVDLAPSKGTVNLTWSRASGKPVNHSTRKEEKQRNGTL
TVTSTLPVGTRDWIEGETYQCRVTHPHLPRALMRSTTKTSGPRAAPEVYA
FATPEWPGSRDKRTLACLIQNFMPEDISVQWLHNEVQLPDARHSTTQPRK
TKGSGFFVFSRLEVTRAEWEQKDEFICRAVHEAASPSQTVQRAVSVNPGK
RSEPKSCDKTHTCPPCPAPELLGGPSVFLFPPKPK
DTLMISRTPEVTCVVVDVSHEDPEVKFNWYVDGVE
VHNAKTKPREEQYNSTYRVVSVLTVLHQDWLNGKE
YKCKVSNKALPAPIEKTISKAKGQPREPQVYTLPP
SREEMTKNQVSLTCLVKGFYPSDIAVEWESNGQPE
NNYKTTPPVLDSDGSFFLYSKLTVDKSRWQQGNVF
SCSVMHEALHNHYTQKSLSLSPGK
HMW-MAA IgE-IgG-Fc C220S Heavy Chain
(SEQ ID NO: 180)
EVQLVQSGGGLVQPGGSLKLSCAVSGFTFSNYWMNWVRQAPGKGLEWVGE
IRLKSNNFGRYYAESVKGRFTISRDDSKNTAYLQMNSLKTEDTAVYYCTS
YGNYVGHYFDHWGQGTLVTVSSASTQSPSVFPLTRCCKNIPSNATSVTLG
CLATGYFPEPVMVTWDTGSLNGTTMTLPATTLTLSGHYATISLLTVSGAW
AKQMFTCRVAHTPSSTDWVDNKTFSVCSRDFTPPTVKILQSSCDGGGHFP
PTIQLLCLVSGYTPGTINITWLEDGQVMDVDLSTASTTQEGELASTQSEL
TLSQKHWLSDRTYTCQVTYQGHTFEDSTKKCADSNPRGVSAYLSRPSPFD
LFIRKSPTITCLVVDLAPSKGTVNLTWSRASGKPVNHSTRKEEKQRNGTL
TVTSTLPVGTRDWIEGETYQCRVTHPHLPRALMRSTTKTSGPRAAPEVYA
FATPEWPGSRDKRTLACLIQNFMPEDISVQWLHNEVQLPDARHSTTQPRK
TKGSGFFVFSRLEVTRAEWEQKDEFICRAVHEAASPSQTVQRAVSVNPGK
RSEPKSSDKTHTCPPCPAPELLGGPSVFLFPPKPK
DTLMISRTPEVTCVVVDVSHEDPEVKFNWYVDGVE
VHNAKTKPREEQYNSTYRVVSVLTVLHQDWLNGKE
YKCKVSNKALPAPIEKTISKAKGQPREPQVYTLPP
SREEMTKNQVSLTCLVKGFYPSDIAVEWESNGQPE
NNKTTPPVLDSDGSFFLYSKLTVDYKSRWQQGNVF
SCSVMHEALHNHYTQKSLSLSPGK
HMW-MAA IgG-IgG-Fc dFcRn Heavy Chain
(SEQ ID NO: 181)
EVQLVQSGGGLVQPGGSLKLSCAVSGFTFSNYWMNWVRQAPGKGLEWVGE
IRLKSNNFGRYYAESVKGRFTISRDDSKNTAYLQMNSLKTEDTAVYYCTS
YGNYVGHYFDHWGQGTLVTVSSASTQSPSVFPLTRCCKNIPSNATSVTLG
CLATGYFPEPVMVTWDTGSLNGTTMTLPATTLTLSGHYATISLLTVSGAW
AKQMFTCRVAHTPSSTDWVDNKTFSVCSRDFTPPTVKILQSSCDGGGHFP
PTIQLLCLVSGYTPGTINITWLEDGQVMDVDLSTASTTQEGELASTQSEL
TLSQKHWLSDRTYTCQVTYQGHTFEDSTKKCADSNPRGVSAYLSRPSPFD
LFIRKSPTITCLVVDLAPSKGTVNLTWSRASGKPVNHSTRKEEKQRNGTL
TVTSTLPVGTRDWIEGETYQCRVTHPHLPRALMRSTTKTSGPRAAPEVYA
FATPEWPGSRDKRTLACLIQNFMPEDISVQWLHNEVQLPDARHSTTQPRK
TKGSGFFVFSRLEVTRAEWEQKDEFICRAVHEAASPSQTVQRAVSVNPGK
RSEPKSCDKTHTCPPCPAPELLGGPSVFLFPPKPK
DTLMASRTPEVTCVVVDVSHEDPEVKFNWYVDGVE
VHNAKTKPREEQYNSTYRVVSVLTVLAQDWLNGKE
YKCKVSNKALPAPIEKTISKAKGQPREPQVYTLPP
SREEMTKNQVSLTCLVKGFYPSDIAVEWESNGQPE
NNYKTTPPVLDSDGSFFLYSKLTVDKSRWQQGNVF
SCSVMHEALHNAYTQKSLSLSPGK
HMW-MAA IgG-IgG-Fc dFcRn C220S Heavy Chain
(SEQ ID NO: 182)
EVQLVQSGGGLVQPGGSLKLSCAVSGFTFSNYWMNWVRQAPGKGLEWVGE
IRLKSNNFGRYYAESVKGRFTISRDDSKNTAYLQMNSLKTEDTAVYYCTS
YGNYVGHYFDHWGQGTLVTVSSASTQSPSVFPLTRCCKNIPSNATSVTLG
CLATGYFPEPVMVTWDTGSLNGTTMTLPATTLTLSGHYATISLLTVSGAW
AKQMFTCRVAHTPSSTDWVDNKTFSVCSRDFTPPTVKILQSSCDGGGHFP
PTIQLLCLVSGYTPGTINITWLEDGQVMDVDLSTASTTQEGELASTQSEL
TLSQKHWLSDRTYTCQVTYQGHTFEDSTKKCADSNPRGVSAYLSRPSPFD
LFIRKSPTITCLVVDLAPSKGTVNLTWSRASGKPVNHSTRKEEKQRNGTL
TVTSTLPVGTRDWIEGETYQCRVTHPHLPRALMRSTTKTSGPRAAPEVYA
FATPEWPGSRDKRTLACLIQNFMPEDISVQWLHNEVQLPDARHSTTQPRK
TKGSGFFVFSRLEVTRAEWEQKDEFICRAVHEAASPSQTVQRAVSVNPGK
RSEPKSSDKTHTCPPCPAPELLGGPSVFLFPPKPK
DTLMASRTPEVTCVVVDVSHEDPEVKFNWYVDGVE
VHNAKTKPREEQYNSTYRVVSVLTVLAQDWLNGKE
YKCKVSNKALPAPIEKTISKAKGQPREPQVYTLPP
SREEMTKNQVSLTCLVKGFYPSDIAVEWESNGQPE
NNYKTTPPVLDSDGSFFLYSKLTVDKSRWQQGNVF
SCSVMHEALHNAYTQKSLSLSPGK
HMW-MAA Kappa Light Chain
(SEQ ID NO: 183)
DIQLTQSPSFLSASVGDRVTITCKASQNVDTNVAWYQQKPGKAPKPLLFS
ASYRYTGVPSRFSGSGSGTDFTLTISSLQPEDFATYFCQQYNSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECVKCK

CHO Transient expression of IgE-IgG (IGEG) variants

Endotoxin-free DNA encoding the differing IGEG constructs were transiently co-transfected into Freestyle™ CHO-S cells (ThermoFisher, Loughborough, UK) using OC-400 processing assemblies and the MaxCyte STX® electroporation system (MaxCyte Inc., Gaithersburg, USA). Following cell recovery, cells were pooled and diluted at 3×10⁶cells/mL into CD Opti-CHO medium (ThermoFisher) containing 8 mM L-Glutamine (ThermoFisher) and 1× Hypoxanthine-Thymidine (ThermoFisher). 24 hours post-transfection, the culture temperature was reduced to 32° C. and 30% (of the starting volume) Efficient Feed B (ThermoFisher), 3.3% FunctionMAX™ TiterEnhancer (ThermoFisher) and 1 mM Sodium Butyrate (Sigma, Dorset, UK) were added. Cultures were fed at Day 7 by the addition of 15% (of the current volume) CHO CD Efficient Feed B (ThermoFisher) and 1.65% FunctionMAX™ TiterEnhancer (ThermoFisher). All transfections were cultured for up to 14 days prior to harvesting supernatants.

Purification and Analysis of IGEG Variants

Following culture harvest, antibody supernatants were filtered to remove remaining cell debris and supplemented with 10× PBS to neutralise pH. The majority of IGEG purifications (including dFcRn IGEGs) were performed using IgE CaptureSelect™ affinity resin (ThermoFisher Scientific) in batch binding mode. Affinity resin was equilibrated in PBS pH 7.2, then incubated with each sample for 2 hours at room temperature with rotation followed by a series of PBS washes. All samples were eluted in 50 mM Sodium Citrate, 50 mM Sodium Chloride pH 3.5 and buffered exchanged into PBS pH 7.2. Samples were quantified by OD₂₈₀ₙₘ using an extinction coefficient (E_{c (0.1%)}) based on the predicted amino acid sequence.

Selected IGEG constructs (e.g. Trastuzumab IGEG containing either Cys220 or Ser220) were purified using Protein A to demonstrate retention of Protein A binding. Following culture harvest, antibody supernatants were filtered to remove remaining cell debris and supplemented with 10× PBS to neutralise pH. Antibodies were then purified from supernatants using 1 mL Hitrap Mab Select PrismA columns (Cytiva, Little Chalfont, UK) previously equilibrated with PBS pH 7.2. Following the sample loading, the columns were washed with PBS pH 7.2 and protein eluted with 0.1 M sodium citrate, pH 3.0. Fractions were collected, and pH adjusted with 1 M Tris-HCl, pH 9.0 followed by buffered exchanged into PBS pH 7.2. Samples were quantified by OD₂₈₀ₙₘ using an extinction coefficient (E_{c (0.1%)}) based on the predicted amino acid sequence.

All IGEG antibody variants were further purified using a HiLoad™ 26/60 Superdex™ 200 pg preparative SEC column (GE Healthcare, Little Chalfont, UK) using PBS pH 7.2 as the mobile phase. Peak fractions from purifications containing monomeric protein were pooled, concentrated and filter sterilised before quantification by A₂₈₀ₙₘ using an extinction coefficient (E_{c (0.1%)}) based on the predicted amino acid sequence.

Purified materials were then analysed by analytical SE-HPLC and SDS-PAGE. Analytical SEC was performed using an Acquity UPLC Protein BEH SEC Column, 200 Å, 1.7 μm, 4.6 mm×150 mm (Waters, Elstree, UK) and an Acquity UPLC Protein BEH SEC guard column 30×4.6 mm, 1.7 μm, 200 Å (Waters, Elstree, UK) connected to a Dionex Ultimate 3000RS HPLC system (ThermoFisher Scientific, Hemel Hempstead, UK). The method consisted of an isocratic elution over 10 minutes and the mobile phase was 0.2 M potassium phosphate pH 6.8, 0.2 M potassium chloride. The flow rate was 0.35 mL/minute. Detection was carried out by UV absorption at 280 nm. Following purification, all IGEG antibody variants were shown to contain >95% monomeric species.

Single Cycle Kinetic Analysis of IGEG Variants to Cognate Antigen

Binding analysis of HMW-MAA IGEG variants to its cognate antigen by Biacore analysis was not possible due to the lack of conformationally appropriate antigens. Binding was, instead, analysed by flow cytometry.

In order to assess the binding of all of the purified Trastuzumab IGEG variants to human Her2 antigen, single cycle kinetic analysis was performed on purified antibodies. Kinetic experiments were performed at 25° C. on a Biacore T200 running Biacore T200 Control software V2.0.1 and Evaluation software V3.0 (Cytiva, Uppsala, Sweden). See FIG. 11 for a schematic of the process.

HBS-EP+ (Cytiva, Uppsala, Sweden), supplemented with 1% BSA (Sigma, Dorset, UK) was used as running buffer as well as for ligand and analyte dilutions. Purified antibodies were diluted in running buffer to 10 μg/mL. At the start of each cycle, antibodies were loaded onto F_{c}2, F_{c}3 and F_{c}4 of an anti-Fab (consisting of a mixture of anti-kappa and anti-lambda antibodies) CMS sensor chip (Cytiva, Little Chalfont, UK). Antibodies were captured at a flow rate of 10 μl/min to give an immobilisation level (R_{L}) of ˜45 RU. The surface was then allowed to stabilise.

Single cycle kinetic data was obtained using recombinant human Her2 antigen (Sino Biological, Beijing, China) as the analyte injected at a flow rate of 40 μL/min to minimise any potential mass transfer effects. A four point, three-fold dilution range from 1.1 nM to 30 nM of antigen in running buffer was used without regeneration between each concentration. The association phases were monitored for 240 seconds for each of the four injections of increasing concentrations of antigen and a single dissociation phase was measured for 600 seconds following the last injection of antigen. Regeneration of the sensor chip surface was conducted using two injections of 10 mM glycine pH 2.1.

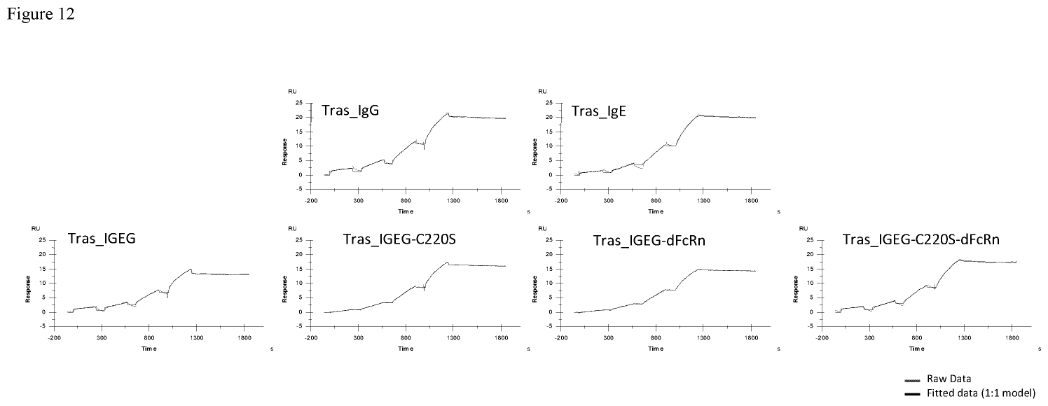

The signal from the reference channel F_{c}1 (no antibody captured) was subtracted from that of F_{c}2, F_{c}3 and F_{c}4 to correct for bulk effect and differences in non-specific binding to a reference surface. The signal from each antibody blank run (antibody captured but no antigen) was subtracted to correct for differences in surface stability (see FIG. 12). Each Trastuzumab construct tested showed similar binding to human Her2 (Table 6).

TABLE 6
Binding parameters of Trastuzumab-IGEG variants to Her2
antigen, as determined using Biacore single cycle kinetics.

| Antibody | kₐ (l/Ms) | k_{d} (l/s) | K_{D} (M) |
|---|---|---|---|
| Trastuzumab_IgG | 1.72E+05 | 7.81E−05 | 4.54E−10 |
| Trastuzumab_IgE | 2.95E+05 | 7.22E−05 | 2.45E−10 |
| Trastuzumab_IGEG | 1.56E+05 | 5.83E−05 | 3.74E−10 |
| Trastuzumab_IGEG-C220S | 1.69E+05 | 4.82E−05 | 2.85E−10 |
| Trastuzumab_IGEG-dFcRn | 1.64E+05 | 4.16E−05 | 2.53E−10 |
| Trastuzumab_IGEG- | 1.38E+05 | 5.82E−05 | 4.23E−10 |
| C220S-dFcRn | | | |

Assessment of IGEG Variant Binding to Human Fc Receptors

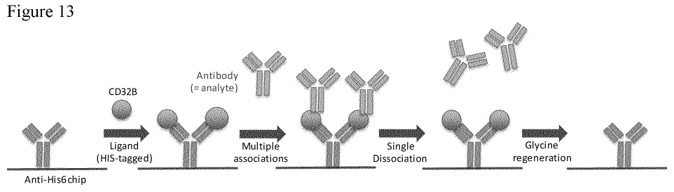

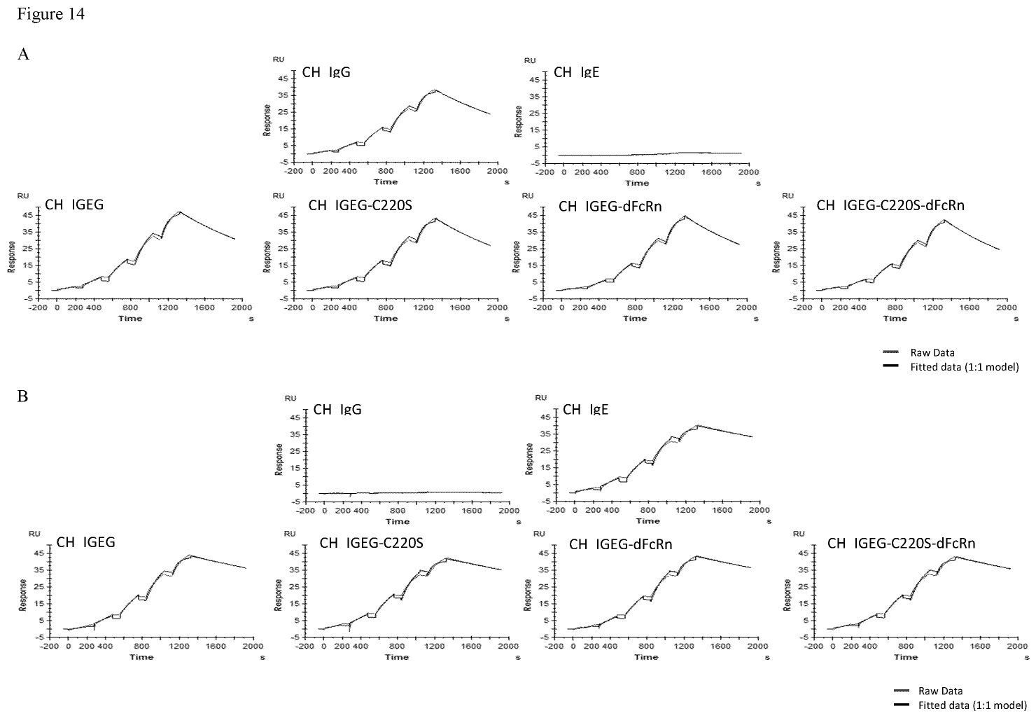

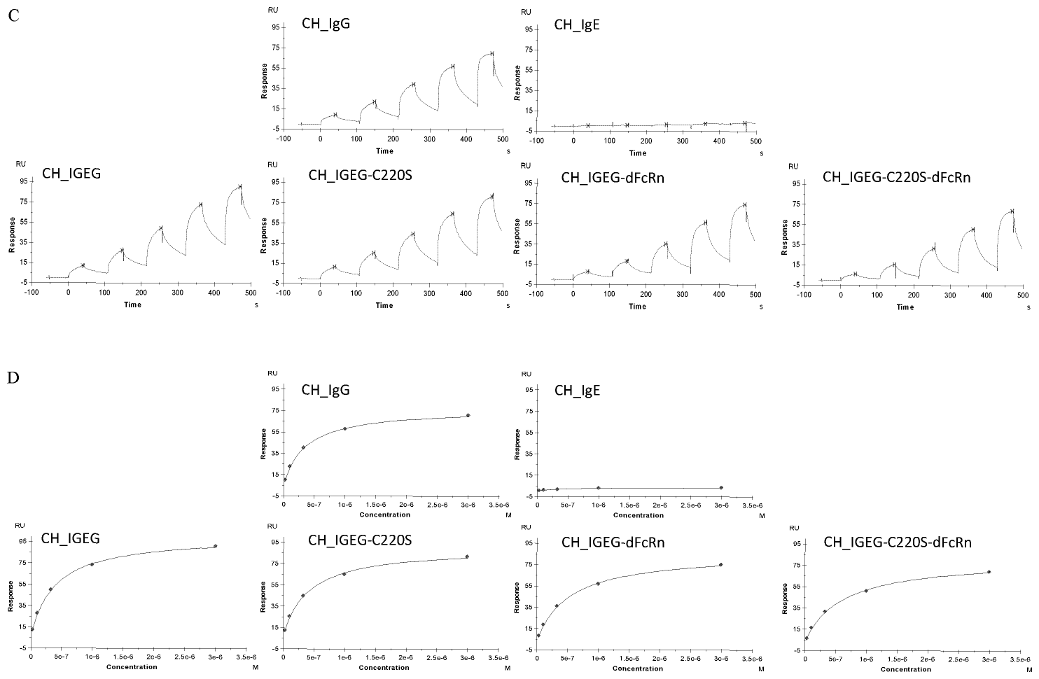

Binding of purified IGEGs to high and low affinity Fc gamma receptors and the high affinity Fc epsilon receptor was assessed by single cycle analysis using a Biacore T200 (serial no. 1909913) instrument running Biacore T200 Evaluation Software V3.0.1 (Uppsala, Sweden) running at a flow rate of 30 μl/min. All of the human Fc gamma receptors (hFcγRT together with the low affinity receptors hFcγRIIIa (both 176F and 176V polymorphisms) and hFcγRIIIb) were obtained from Sino Biological (Beijing, China) and hFcεR1 was obtained from R&D Systems (Minneapolis, USA). FcRs were captured on a CM5 sensor chip pre-coupled using a His capture kit (Cytiva, Uppsala, Sweden) using standard amine chemistry. A schematic detailing the assay used to assess antibody binding to Fc gamma receptors can be found in FIG. 13.

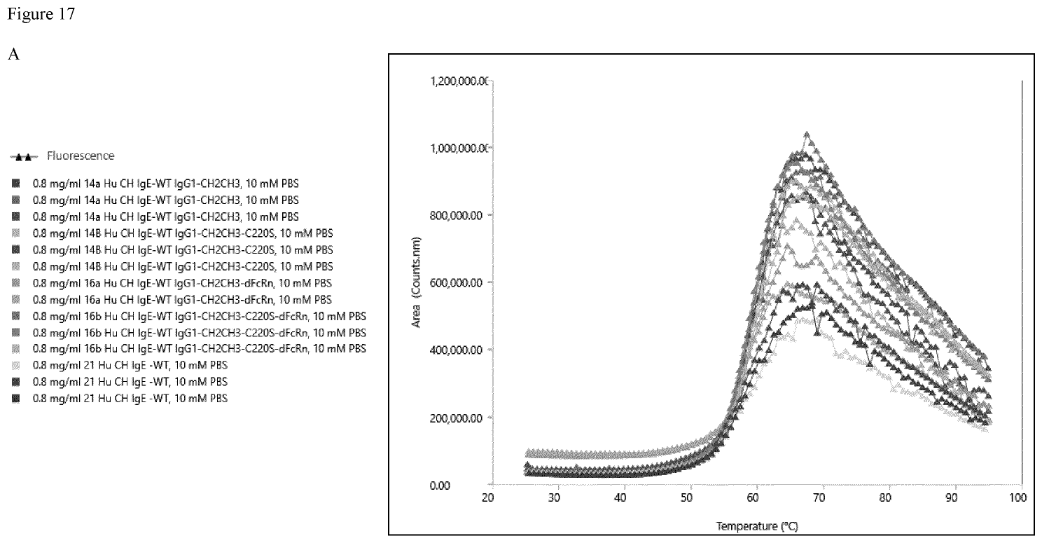

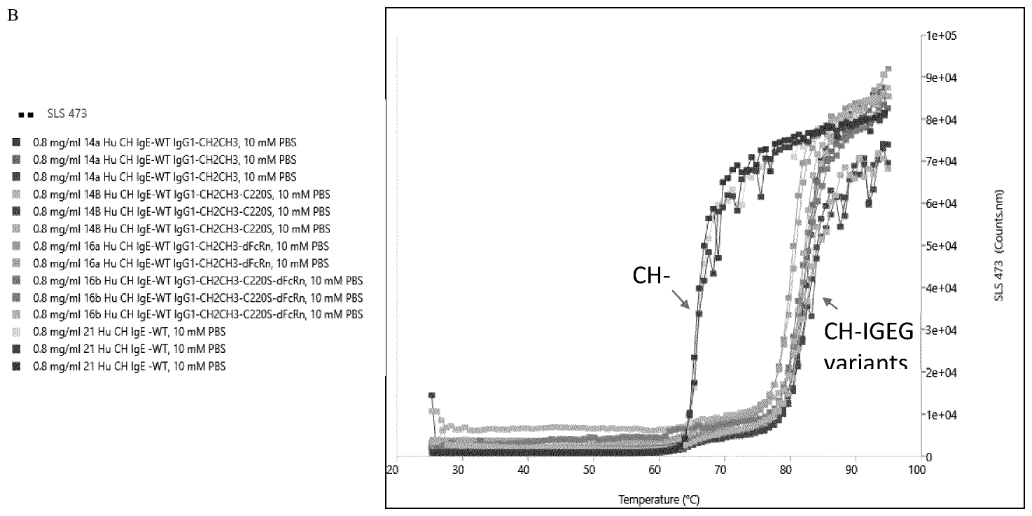

At the start of each cycle His-tagged Fc receptors diluted in HEPES buffered saline containing 0.05% v/v Surfactant P20 (HBS-P+) were loaded to a specified RU level (Table 7). A five point, three-fold dilution range of test antibody without regeneration between each concentration was used for each receptor tested. The target RU loaded for each Fc receptor, association and dissociation times used for test antibody binding together with the concentration range used for each test antibody are shown in (Table 7). In all cases, antibodies were passed over the chip in increasing concentrations followed by a single dissociation step. Following dissociation, the chip was regenerated with two injections of Glycine pH 1.5. The signal from the reference channel F_{c}1 (blank) was subtracted from that of the F_{c} loaded with receptor to correct for differences in non-specific binding to the reference surface. High affinity interactions were analysed using 1:1 fit (see FIGS. 17a and 17b for example data), whereas the low affinity interactions were analysed using a steady state model (see FIGS. 17c and 17d for example data). Table 8 shows a summary of the data obtained. IGEG variants bound to both the Fcgamma receptors tested and to Fcepsilon receptor. IgG control found to the Fcgamma receptors and not to Fcepsilon, whereas conversely, IgE control found to the Fcepsilon receptor and not to the Fcgamma receptors tested.

TABLE 7
Experimental parameters (as defined within the experimental setup)
used for the assessment of binding of IGEG variants to Fc gamma
and Fc epsilon receptors using Biacore single cycle kinetics.

| | Binding | RU | Concentration | Association | Dissociation | |
|---|---|---|---|---|---|---|
| Name | affinity | loaded | Range (nM) | (s) | (s) | Analysis |
| FcγRI | High | 30 | 0.411 to 33.33 | 200 | 600 | 1:1 |
| | | | | | | Affinity |
| FcγRIIIA_{176Phe} | Low | 20 | 98.8 to 8000 | 45 | 25 | Steady |
| | | | | | | State |
| FcγRIIIA_{176Val} | Low | 20 | 98.8 to 8000 | 45 | 25 | Steady |
| | | | | | | State |
| FcγRIIIB | Low | 60 | 98.8 to 8000 | 45 | 25 | Steady |
| | | | | | | State |
| Fcε Ria | High | 30 | 0.411 to 33.33 | 200 | 600 | 1:1 |
| | | | | | | Affinity |

TABLE 8
1:1 (FcgRI and FceRIa) or Steady state affinity (FcγRIIIA_{176Phe}, FcγRIIIA_{176Val}
and FcγRIIIB) summary data for the binding of Trastuzumab and HMW-MAA-IGEG
variants to Fc gamma and Fc epsilon receptors, as determined using Biacore single cycle kinetics.

| | | | Human CD16A | | Human CD16A | | | | | |
|---|---|---|---|---|---|---|---|---|---|---|
| | Human CD64 | | 176 Phe | | 176 Val | | Human CD16B | | | |
| | (FcgRI) | | (FcγRIIIA_{176Phe}) | | (FcγRIIIA_{176Val} ) | | (FcγRIIIB) | | Human FceRIa | |
| Antibody | K_{D} | Relative | K_{D} | Relative | K_{D} | Relative | K_{D} | Relative | K_{D} | Relative |
| | (M) | binding | (M) | Binding | (M)* | Binding | (M)* | Binding | (M) | binding |
| Control IgG1 | 2.47E−09 | ++++ | 2.19E−06 | ++ | 7.20E−07 | +++ | 3.89E−06 | ++ | — | — |
| Trastuzumab_ | — | — | — | — | — | — | — | — | 4.11E−10 | +++++ |
| IgE | | | | | | | | | | |
| Trastuzumab_ | 2.35E−09 | ++++ | 6.64E−07 | +++ | 2.20E−07 | +++ | 1.70E−06 | ++ | 5.38E−10 | +++++ |
| IGEG | | | | | | | | | | |
| Trastuzumab_ | 2.37E−09 | ++++ | 6.95E−07 | +++ | 2.33E−07 | +++ | 1.73E−06 | ++ | 5.57E−10 | +++++ |
| IGEG-C220S | | | | | | | | | | |
| Trastuzumab_ | 3.27E−09 | ++++ | 1.32E−06 | ++ | 4.38E−07 | +++ | 2.54E−06 | ++ | 5.65E−10 | +++++ |
| IGEG-dFcRn | | | | | | | | | | |
| Trastuzumab_ | 3.55E−09 | ++++ | 1.42E−06 | ++ | 4.88E−07 | +++ | 3.08E−06 | ++ | 5.77E−10 | +++++ |
| IGEG-C220S- | | | | | | | | | | |
| dFcRn | | | | | | | | | | |
| HMW-MAA_ | 2.65E−09 | ++++ | 8.42E−07 | +++ | 3.32E−07 | +++ | 2.02E−06 | ++ | — | — |
| IgG | | | | | | | | | | |
| HMW-MAA_ | — | — | — | — | — | — | — | — | 5.08E−10 | +++++ |
| IgE | | | | | | | | | | |
| HMW-MAA_ | 1.63E−09 | ++++ | 6.65E−07 | +++ | 3.86E−07 | +++ | 1.48E−06 | ++ | 5.99E−10 | +++++ |
| IGEG | | | | | | | | | | |
| HMW-MAA_ | 1.67E−09 | ++++ | 7.75E−07 | +++ | 4.00E−07 | +++ | 1.87E−06 | ++ | 4.93E−10 | +++++ |
| IGEG-C220S | | | | | | | | | | |
| HMW-MAA_ | 2.02E−09 | ++++ | 9.60E−07 | +++ | 5.35E−07 | +++ | 2.03E−06 | ++ | 5.56E−10 | +++++ |
| IGEG-dFcRn | | | | | | | | | | |
| HMW-MAA_ | 2.18E−09 | ++++ | 1.16E−06 | +++ | 6.01E−07 | +++ | 2.78E−06 | ++ | 5.35E−10 | +++++ |
| IGEG-C220S- | | | | | | | | | | |
| dFcRn | | | | | | | | | | |

Assessment of IGEG Variant Binding to Human FcRn

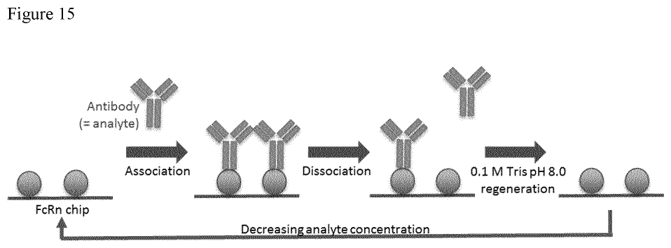

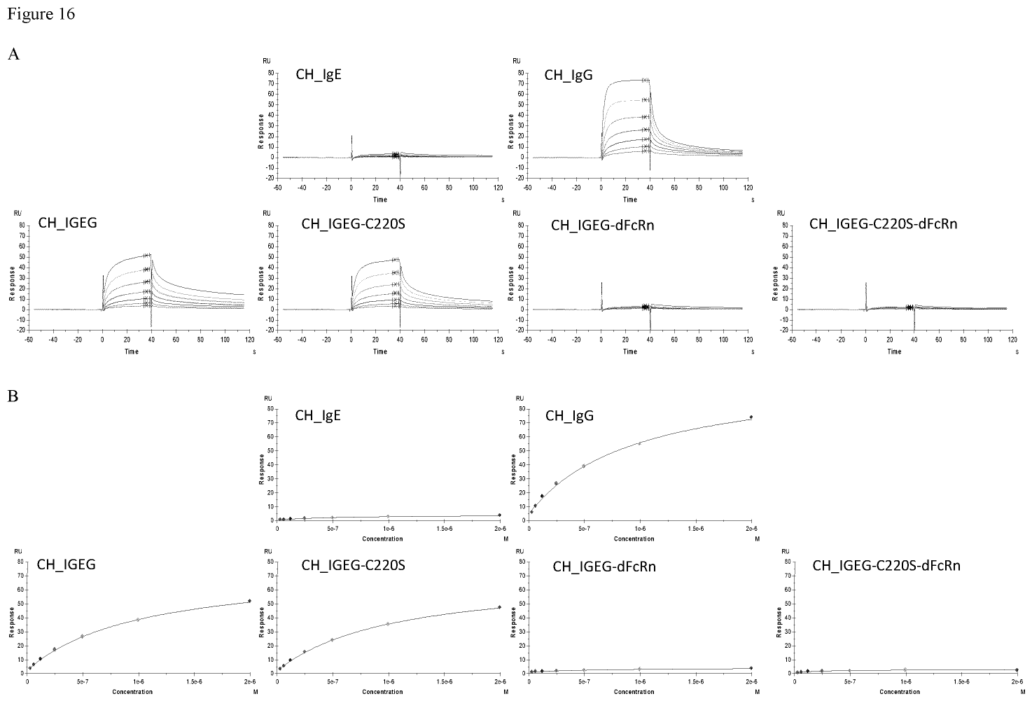

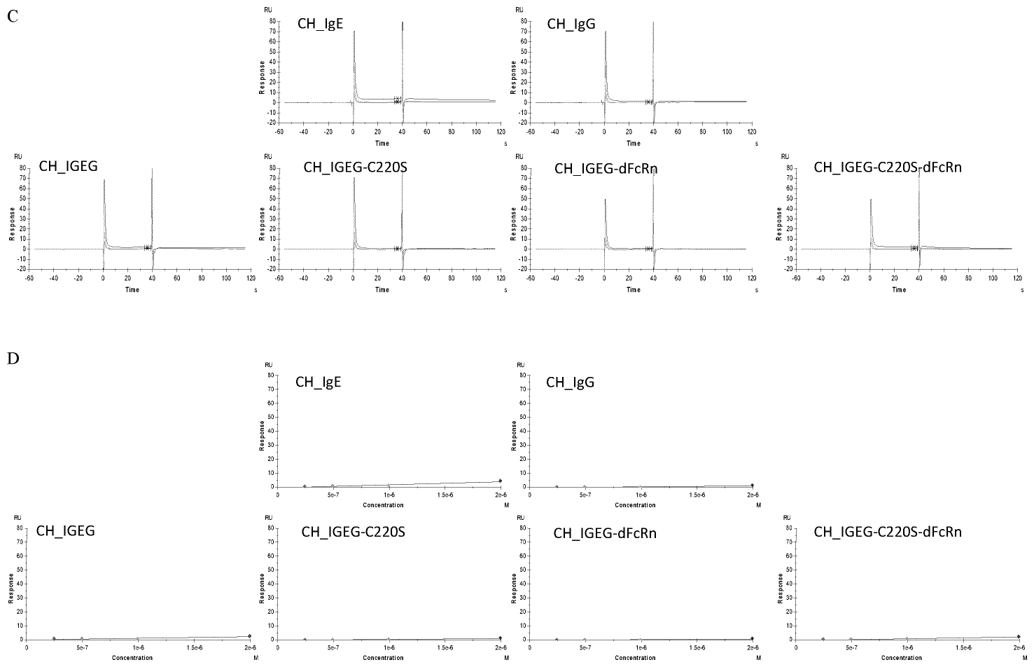

The binding of the purified antibodies to FcRn was assessed by steady state affinity analysis using a Biacore T200 (serial no. 1909913) instrument running Biacore T200 Evaluation Software V3.0.1 (Uppsala, Sweden). hFcRn (Sino Biological, Beijing, China) was coupled onto a Series S CM5 (carboxymethylated dextran) sensor chip (Cytiva, Uppsala, Sweden) at 10 μg/mL in sodium acetate pH 5.5 using standard amine coupling. Purified HMW-MAA antibodies were titrated in a seven point, two fold dilution from 31.25 nM to 2000 nM in PBS containing 0.05% Polysorbate 20 (P20) at pH 6.0 or a four three point, two-fold dilution from 250 nM to 2000 nM in PBS containing 0.05% Polysorbate 20 (P20) at pH 7.4. Antibodies were passed over the chip with increasing concentrations at a flow rate of 30 μl/min and at 25° C. The injection time was 40 s per concentration and the dissociation time was 75 s. Following a single dissociation, the chip was regenerated with 0.1 M Tris pH 8.0. FIG. 15 shows a schematic of the assay used to assess used to assess antibody binding to FcRn. Interactions were analysed using a steady state model (see FIGS. 16a to 16d for example data). Table 9 shows a summary of the data obtained. IGEG variants bound to FcRn at pH 6.0 with the exception of those in which the FcRn binding site has been removed (dFcRn) and which failed to bind FcRn. IgG control found to FcRn as expected whereas IgE did not show any binding to FcRn.

TABLE 9
Steady state affinity summary data for the binding of
Trastuzumab and HMW-MAA-IGEG variants to FcRn at pH 6.0
or pH 7.4, as determined using Biacore single cycle kinetics.

| | FcRn pH 6.0 | FcRn pH 7.4 |
|---|---|---|
| Antibody | K_{D} (M) | K_{D} (M) |
| Control IgG1 | 6.12E−07 | — |
| Trastuzumab_IgE | — | — |
| Trastuzumab_IGEG | 4.77E−07 | — |
| Trastuzumab_IGEG-C220S | 5.06E−07 | — |
| Trastuzumab_IGEG-dFcRn | — | — |
| Trastuzumab_IGEG-C220S-dFcRn | — | — |
| HMW-MAA_IgG | 9.58E−07 | — |
| HMW-MAA_IgE | — | — |
| HMW-MAA_IGEG | 1.02E−06 | — |
| HMW-MAA_IGEG-C220S | 1.05E−06 | — |
| HMW-MAA_IGEG-dFcRn | — | — |
| HMW-MAA_IGEG-C220S-dFcRn | — | — |

UNcle Biostability Platform Analysis of IGEG Variants

IGEG variants were analysed for thermal stability using the UNcle biostability platform (Unchained labs, Pleasanton, USA). Thermal ramp stability experiments (Tm and Tagg) are well established methods for ranking proteins and formulations for stability. A protein's denaturation profile provides information about its thermal stability and represents a structural ‘fingerprint’ for assessing structural and formulation buffer modifications. A widely used measure of the thermal structural stability of a protein is the temperature at which it unfolds from the native state to a denatured state. For many proteins, this unfolding process occurs over a narrow temperature range and the mid-point of this transition is termed ‘melting temperature’ or ‘Tm’. To determine the melting temperature of a protein, UNcle measures the fluorescence of Sypro Orange (which binds to exposed hydrophobic regions of proteins) as the protein undergoes conformational changes.

Samples for each variant were formulated in PBS and Sypro Orange at a final concentration of 0.8 mg/mL. 9 μL of each sample mixture was loaded in duplicate into UNi microcuvettes.

Samples were subjected to a thermal ramp from 25-95° C., with a ramp rate of 0.3° C./minute and excitation at 473 nm. Full emission spectra were collected from 250-720 nm, and the area under the curve between 510-680 nm was used to calculate the inflection points of the transition curves (Tₒₙₛₑₜ and Tₘ). Monitoring of static light scattering (SLS) at 473 nm allowed the detection of protein aggregation, and T_{agg} (onset of aggregation) was calculated from the resulting SLS profiles. Data analysis was performed using UNcle™ software version 4.0 and summarised in Table 10. Tm1 values were broadly consistent within each set of variants and between IgE and IGEG variants (FIG. 17a), however, the IGEG variants showed a significant improvement in static light scattering profile compared to the equivalent IgE variants alone (FIG. 17b).

TABLE 10
Summary of thermal stability values for the purified IGEG
variants, as determined using the UNcle biostability platform.

| | | | T_{agg} |
|---|---|---|---|
| | Tₘ1 | Tₒₙₛₑₜ | (° C.) |
| | (° C.) | (° C.) | (473 nm) |
| Antibody | Average | Average | Average |
| Trastuzumab IGEG | 57.5 | 50.4 | 76.7 |
| Trastuzumab IGEG-C220S | 57.6 | 50.3 | 78.2 |
| Trastuzumab IGEG-dFcRn | 58.1 | 51.7 | 76.7 |
| Trastuzumab IGEG-C220S-dFcRn | 57.5 | 51.5 | ND |
| Trastuzumab IgE-WT | 56.6 | 45.7 | 66 |
| HMW-MAA IGEG | 59.4 | 52.0 | 77.4 |
| HMW-MAA IGEG-C220S | 59.1 | 51.3 | 77.6 |
| HMW-MAA IGEG-dFcRn | 58.9 | 50.0 | 75.7 |
| HMW-MAA IGEG-C220S-dFcRn | 59.5 | 51.4 | 76.7 |
| HMW-MAA IgE-WT | 57.2 | 48.2 | 63.5 |

### Example 6

### Assessment of IGEG variant binding to A375 cells

Binding of the antibody variants detailed in Examples 4 and 5 to HMW-MAA was assessed using A375 cells, which express HMW-MAA (CSPG4).

Method

Harvesting A375 Cells

A375 cells were cultured using standard methods. When A375 cells were confluent, the cells were harvested. In brief, cells were washed with PBS before incubation with TrypLE™ at 37° C. for 10 minutes to detach the cells from the flask. Cells were resuspended in 10 mL of media and centrifuged for 3 minutes at 250 g. Cells were then resuspended in 1 mL FACS buffer and counted on the Cellometer® to determine the cell number and viability. Following this, cells were diluted to 1×10⁶ cells per mL with FACS buffer, and 100 μL of this cell suspension plated per well on a plate.

Binding Assay

Binding of purified IGEGs to A375 cells (ATCC, Virginia, US) was assessed by flow cytometry using a Attune® N×T Acoustic Focusing Cytometer running Attune Software V3.1.2 (ThermoFisher Scientific, Loughborough, UK). A375 cells were incubated with the primary antibodies (as described in Example 5) for 30 min at 4° C. followed by incubation with FITC conjugated Goat anti-human anti-IgG or IgE secondary antibodies (Vector Laboratories, California, US) at 10 μg/m1 for a further 30 minutes at 4° C. Cells were washed and resuspended in FACS buffer and then acquired on the Attune® N×T Acoustic Focusing Cytometer. The data was analysed using FlowJo™ Software Version 10 (Becton, Dickinson and Company, New Jersey, US) and GraphPad Prism 8 (GraphPad Software, California, US).

Results

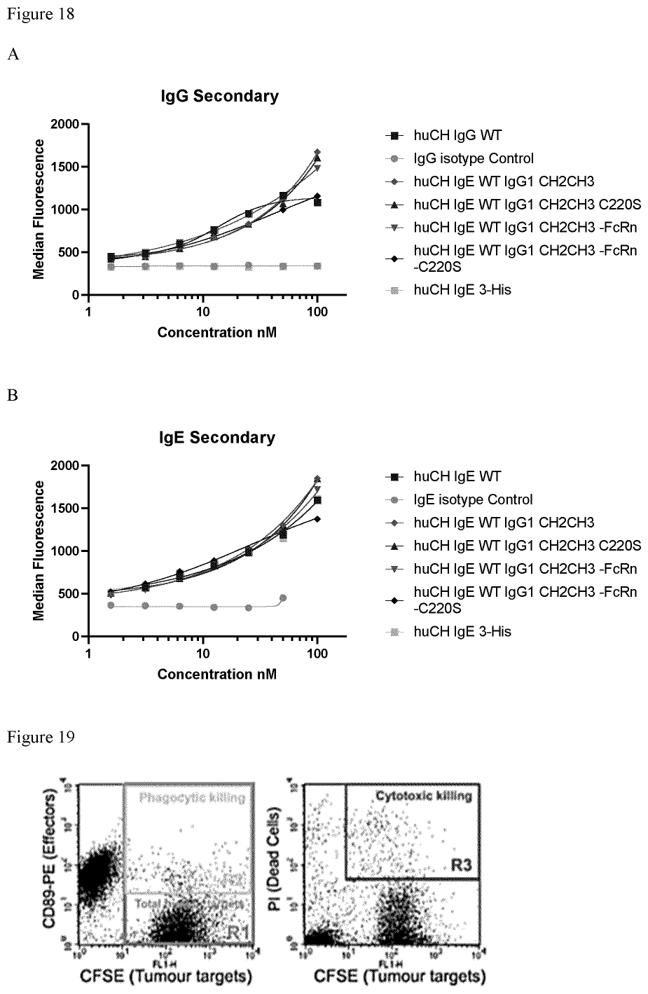

As demonstrated in FIGS. 18a and 18b, all HMW-MAA antibodies and variants bound to A375 cells.

### Example 7

### ADCC and ADCP assays

Assays were performed to determine the effects of the described antibodies on levels of both antibody-dependent cell-mediated phagocytosis (ADCP) and antibody dependent cell-mediated cytotoxicity (ADCC), the two main mechanisms by which immune effector cells can kill tumour cells. The antibody variants described in Example 5 were compared to Trastuzumab IgE and Herceptin IgG antibodies.

Method

ADCC and ADCP assays were performed using methods similar to those existing in the art (for example, see Three-colour flow cytometric method to measure antibody-dependent tumour cell killing by cytotoxicity and phagocytosis. J Immunol Methods. 2007 Jun 30;323(2):160-71) using U-937 effector cells and SK-BR-3 target cells.

The day prior to performing the assay, Her2-expressing tumour cells (SK-BR-3) were stained. To do this, SK-BR-3 cells were detached from the plate using TrypLE, washed with complete RPMI media (RPMI 1640 media supplemented with pen/strep and 10% HI FBS) before adding to serum-free HBSS. 0.75 μuL 0.5 mM carboxyflourescein succinimidyl ester (CSFE) in HBSS was added per 1×10⁶ cells and cells incubated at 37° C. for 10 minutes. After washing, cells were plated and incubated overnight.

The next day, U-937 effector cells were passaged, counted using Trypan blue and resuspended in complete RPMI media to provide 1.5×10⁶ cells per mL. The CFSE-labelled SK-BR-3 cells were detached by TrypLE treatment, washed, counted, and re-suspended in complete RPMI media to provide 0.5×10⁶ cells per mL. The Trastuzumab IgE, Herceptin IgG, Trastuzumab-IGEG, Trastuzumab-IGEG-C220S, and IgG isotype antibodies detailed in Example 5 were then diluted to a starting concentration of 120 nM and then serially diluted by a factor of six. 25 μL of each antibody dilution was added to a 96-well plate in duplicate along with 50 μL of the SK-BR-3 cell suspension (equivalent to 25000 cells) and 25 μL of the U-937 effector cell suspension (equivalent to 37500 cells). Appropriate control wells lacking one or more of: CSFE staining, U-397 cells, SK-BR-3 cells, viable SK-BR-3 cells (replaced by heat-shocked SK-BR-3 cells) or test antibody were included in the assay. The plate was then incubated for 3 hours at 37° C., centrifuged and washed with FACS buffer (PBS +2% FCS) twice before resuspending in 100 μL FACS with 2 μL CD89 APC-conjugated labelling antibody. Control wells were resuspended in FACS buffer alone. After 30 minutes at 4° C., the plate was centrifuged and washed again with FACS buffer twice before resuspending the cells in 100 μL FACS buffer containing propidium iodide (PI) stain (5 μL per 100 μL). Control wells were resuspended in FACS buffer and incubated for 15 minutes at room temperature.

50,000 cells/tube were then acquired on the Attune™ N×T Acoustic Focusing Cytometer. Compensation was set-up using control wells. R1, R2, R3 gating was applied in analysis software (Flow Jo) (FIG. 19) and cell counts obtained per gate. Calculations were then performed to determine the cytotoxicity (ADCC) or phagocytic (ADCP) activity.

Results

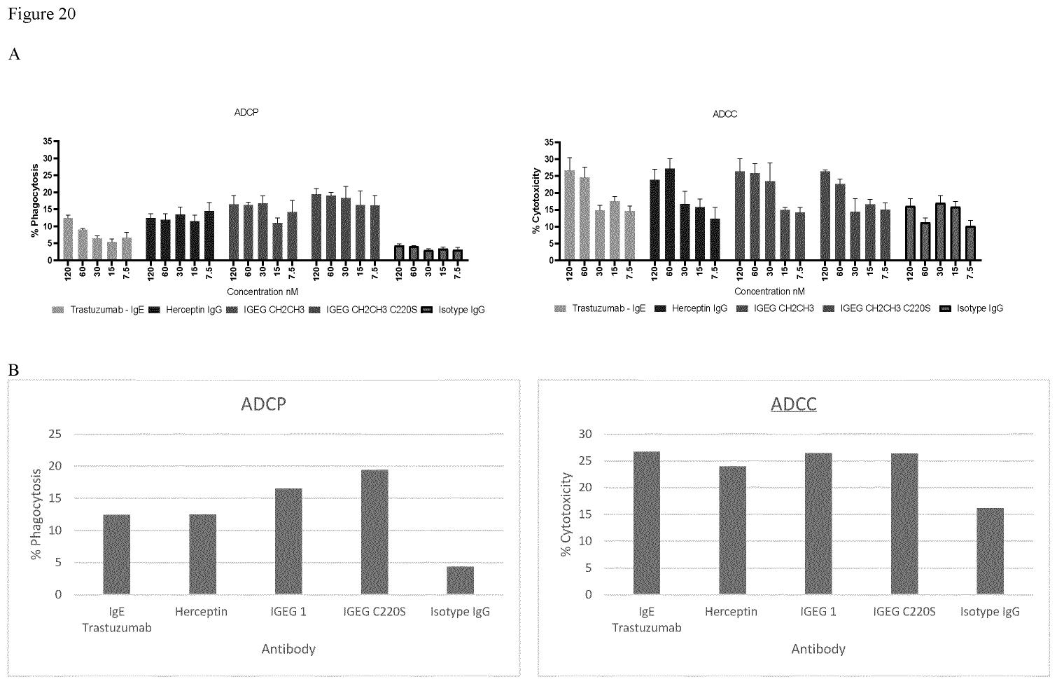

As demonstrated in FIG. 20, the Trastuzumab-IGEG (IGEG-CH2CH3) antibody appears to result in higher levels of phagocytosis than the Herceptin IgG and Trastuzumab IgE antibodies across all concentrations tested (120-7.5 nM). The Trastuzumab-IGEG-C200S (IGEG-CH2CH3-C220S) antibody appears to result in higher levels of phagocytosis than the Herceptin IgG and Trastuzumab IgE antibodies. In addition, the results demonstrate that the Trastuzumab IgE, Herceptin IgG and both IGEG antibodies had comparable effects on cytotoxicity.

The present application claims priority from UK patent application no. 1914165.4, filed 1 Oct. 2019, UK patent application no. 1917059.6, filed 22 Nov. 2019 and UK patent application no. 2008248.3, filed 2 Jun. 2020, the contents of which are incorporated herein by reference. All publications mentioned in the above specification are herein incorporated by reference. Various modifications and variations of the described embodiments of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A hybrid antibody that binds an FCC receptor and neonatal Fc receptor (FcRn).

2. The hybrid antibody according to claim 1, comprising one or more heavy chain constant domains, or variants or functional fragments thereof, derived from an IgE antibody.

3. The hybrid antibody according to claim 1 or claim 2, comprising at least a Cε3 domain, or a variant or functional fragment thereof.

4. The hybrid antibody according to any preceding claim, comprising at least Cε2, Cε3 and Cε4 domains, or variants or functional fragments thereof.

5. The hybrid antibody according to any preceding claim, wherein the antibody comprises all or part of a binding site for FcRn, or variants or functional fragments thereof, derived from an IgG antibody.

6. The hybrid antibody according to any preceding claim, wherein FcRn binding is provided by one or more amino acid substitutions in at least one Fc domain of a tetrameric IgE.

7. The hybrid antibody according to claim 6, comprising:
(i) at least one amino acid substitution in Cε3 of IgE;
(ii) at least one amino acid substitution in Cε4 of IgE; and/or
(iii) one amino acid substitution in Cε3 and two amino acid substitutions in Cε4 of IgE.

8. The hybrid antibody according to claim 6 or claim 7, wherein the amino acid substitutions in the IgE comprise non-native histidine residues present at a corresponding position in an IgG.

9. The hybrid antibody according to any preceding claim, comprising an IgE antibody comprising one, two or three heterologous histidine residues that confer FcRn-binding.

10. The hybrid antibody according to any one of claims 6 to 9, wherein threonine is substituted for histidine in Loop 2 of Cε3 of IgE.

11. The hybrid antibody according to any one of claims 6 to 10, wherein a serine is substituted for histidine and glutamine is substituted for histidine in Loop 3 of Cε4 of IgE.

12. The hybrid antibody according to any preceding claim, comprising:
(i) a variant IgE Cε3 domain comprising a histidine residue at position 78;
(ii) a variant IgE Cε4 domain comprising a histidine residue at position 95 and/or 98.

13. The hybrid antibody according to any preceding claim, comprising:
(i) an IgE Cε3 domain having at least 85%, 90%, 95% or 99% sequence identity to SEQ ID NO:2, and comprising the mutation T78H; and/or
(ii) an IgE Cε4 domain having at least 85%, 90%, 95% or 99% sequence identity to SEQ ID NO:3, and comprising the mutation S95H and/or Q98H.

14. The hybrid antibody according to any preceding claim, comprising:
(i) an IgE Cε3 loop sequence as defined in SEQ ID NO:31; and/or
(ii) an IgE Cε4 loop sequence as defined in SEQ ID NO:32 or 33.

15. The hybrid antibody according to any preceding claim, comprising an amino acid sequence having at least 85%, 90%, 95% or 99% sequence identity with SED ID NO:26, and comprising a histidine residue at position(s) 78, 203 and/or 206 of SEQ ID NO:26.

16. The hybrid antibody according to any preceding claim, wherein the antibody comprises an amino acid sequence having at least 85%, 90%, 95% or 99% sequence identity with SEQ ID NO:1.

17. The hybrid antibody according to any preceding claim, comprising an amino acid sequence having at least 85%, 90%, 95% or 99% sequence identity with the sequence of SEQ ID NO:34, and comprising a histidine residue at position(s) 408, 533 and/or 536 of SEQ ID NO:34.

18. The hybrid antibody according to any preceding claim, wherein binding of the antibody to FcRn is pH-dependent, preferably wherein the antibody has a higher affinity for FcRn at pH 6.0 than at pH 7.4.

19. The hybrid antibody of any preceding claim, wherein the antibody binds specifically to a cancer antigen.

20. A pharmaceutical composition comprising a hybrid antibody as defined in any preceding claim and a pharmaceutically acceptable excipient, diluent or carrier.

21. A hybrid antibody or pharmaceutical composition as defined in any preceding claim for use in preventing or treating cancer.

22. A nucleic acid that encodes a heavy chain of a hybrid antibody, wherein the heavy chain comprises an amino acid sequence having at least 85%, 90%, 95% or 99% sequence identity to (i) SEQ ID NO:1 and SEQ ID NO:26; and/or (ii) SEQ ID NO:34.

23. An expression vector comprising the nucleic acid as defined in claim 22, optionally wherein (i) the vector is a CHO vector and/or (ii) the nucleic acid is operably linked to a promoter suitable for expression in mammalian cells.

24. A host cell comprising a recombinant nucleic acid encoding a hybrid antibody as defined in any one of claims 1 to 19.

25. The host cell according to claim 24, comprising the nucleic acid sequence as defined in claim 22 or the vector as defined in claim 23.

26. A method of producing a hybrid antibody as defined in any one of claims 1 to 19 comprising culturing host cells as defined in claim 24 or claim 25 under conditions for expression of the antibody and recovering the antibody or a fragment thereof from the host cell culture.

27. A hybrid antibody according to any preceding claim, comprising a light chain amino acid sequence having at least 85%, 90%, 95% or 99% sequence identity with the sequence of SEQ ID NO:35.

28. The hybrid antibody according to any preceding claim, comprising an amino acid sequence having at least 85%, 90%, 95% or 99% sequence identity with the sequence of SEQ ID NO:186, and comprising a histidine residue at position(s) 411, 536 and/or 539 of SEQ ID NO:186.

29. The hybrid antibody according to any preceding claim, comprising an amino acid sequence having at least 85%, 90%, 95% or 99% sequence identity with the sequence of SEQ ID NO:188, and comprising a histidine residue at position(s) 410, 535 and/or 538 of SEQ ID NO:188.

30. A hybrid antibody according to any preceding claim, comprising a light chain amino acid sequence having at least 85%, 90%, 95% or 99% sequence identity with the sequence of SEQ ID NO:187 or 189.
